(19) United States
(12) Patent Application Publication
(10) Pub. No.: US 2026/0355235 A1
(43) Pub. Date: Nov. 12, 2026

(54) INHIBITION OF FORMIC ACID METABOLISM AS A TREATMENT FOR BACTERIAL VAGINOSIS

(71) Applicant: Fred Hutchinson Cancer Center, Seattle, WA (US)

(72) Inventors: Elliot Lee, Seattle, WA (US); David Fredricks, Seattle, WA (US)

(73) Assignee: Fred Hutchinson Cancer Center, Seattle, WA (US)

(21) Appl. No.: 19/669,033

(22) Filed: May 6, 2026

Related U.S. Application Data
(60) Provisional application No. 63/801,000, filed on May 6, 2025.

(51) Int. Cl. A61K 33/42; A61K 45/06; A61P 31/04

(52) U.S. Cl. CPC A61K 33/42; A61K 45/06; A61P 31/04

(57) ABSTRACT

Disclosed herein are methods for treating bacterial vaginosis (BV) or reducing BV recurrence. In one aspect, the methods include administering an inhibitor of formic acid metabolism in BV-associated bacteria (BVAB), thereby reducing growth, metabolic activity, or both, of the BVAB. In some embodiments, the inhibitor is an inhibitor of pyruvate formate lyase.

### CROSS-REFERENCE TO RELATED APPLICATION

This application claims the benefit of U.S. Provisional Application No. 63/801,000, filed May 6, 2025, the disclosure of which is incorporated herein by reference in its entirety.

### STATEMENT OF GOVERNMENT LICENSE RIGHTS

This invention was made with government support under AI061628 awarded by the National Institutes of Health. The government has certain rights in the invention.

### BACKGROUND

Bacterial vaginosis (BV) is a common condition associated with disruption of the normal vaginal microbiota. In many healthy individuals, the vaginal microbiota is dominated by commensal Lactobacillus species, which help maintain a protective vaginal environment. In BV, the abundance of Lactobacillus species is reduced, and diverse anaerobic and facultative bacteria increase in abundance, resulting in a polymicrobial community associated with elevated pH, discharge, odor, and increased risk of adverse reproductive and infectious outcomes.

Current treatments for BV generally rely on antibiotics. Although antibiotics can reduce symptoms and temporarily suppress BV-associated bacteria (BVAB), recurrence remains common. One reason for this recurrence may be that BVAB form complex, interbacterial relationships supported by shared nutrients, metabolites, and complementary metabolic pathways. Antibiotic treatments that broadly suppress bacteria may also affect beneficial commensal organisms and may not selectively disrupt the metabolic processes that allow BVAB to persist.

Accordingly, there remains an unmet need for improved methods of treating BV and reducing recurrence of BV. In particular, there is a need for therapies that specifically target metabolic pathways used by BVAB, while reducing effects on beneficial commensal bacteria, such as Lactobacillus species.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an aspect, the present disclosure provides a method of treating bacterial vaginosis (BV) or reducing a recurrence of BV in a subject, the method comprising administering to the subject a therapeutically effective amount of an inhibitor of formic acid metabolism in one or more BV-associated bacteria, wherein the inhibitor prevents or reduces growth, metabolic activity, or a combination thereof, of the one or more BV-associated bacteria.

In some embodiments, the therapeutically effective amount is an amount effective to inhibit formic acid production, formic acid consumption, or a combination thereof, by the one or more BV-associated bacteria.

In some embodiments, the inhibitor is an inhibitor of pyruvate formate lyase (PFL).

In some embodiments, the inhibitor is an inhibitor of formate dehydrogenase (FDH).

In some embodiments, the inhibitor comprises a formate analog.

In some embodiments, the inhibitor comprises hypophosphite or a pharmaceutically acceptable salt thereof.

In some embodiments, the one or more BV-associated bacteria comprise Gardnerella vaginalis, Gardnerella piotii, Gardnerella leopoldii, Gardnerella swidsinskii, Fannyhessea vaginae, Amygdalobacter indicium, Amygdalobacter nucleatus, Megasphaera lornae, Megasphaera vaginalis, Megasphaera curtisii, Megasphaera mulieris, Prevotella bivia, Sneathia vaginalis, Umbribacter vaginalis, or a combination thereof.

In some embodiments, the inhibitor selectively prevents or reduces growth, metabolic activity, or a combination thereof, of the one or more BV-associated bacteria relative to one or more Lactobacillus species.

In some embodiments, the inhibitor does not impact a growth of the one or more Lactobacillus species.

In some embodiments, the one or more Lactobacillus species comprise Lactobacillus crispatus, Lactobacillus jensenii, Lactobacillus gasseri, Lactobacillus iners, or a combination thereof.

In some embodiments, the inhibitor is formulated as a topical composition for intravaginal application.

In some embodiments, the inhibitor is administered intravaginally.

In some embodiments, the inhibitor is administered in combination with an antibiotic for treating BV. The antibiotic can be, for example, metronidazole, clindamycin, secnidazole, tinidazole, and the like.

In some embodiments, the inhibitor is administered after completion of an antibiotic treatment for BV.

In some embodiments, the inhibitor is administered prophylactically to a subject at risk of BV recurrence.

In some embodiments, administration of the inhibitor promotes restoration of a vaginal microbiota dominated by one or more Lactobacillus species.

In some embodiments, administration of the inhibitor reduces vaginal malodor associated with BV.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C are box plots that show signs of increased bacterial concentration and metabolism in bacterial vaginosis (BV). FIG. 1A shows a box plot indicating a total bacterial load as measured by 16S rRNA gene copy number for BV-negative (BV−) and BV-positive (BV+) vaginal samples. FIG. 1B shows a number of significant bacterial peptide-spectrum matches (PSMs) identified in the BV− and BV+ vaginal samples. FIG. 1C shows a number of significant human PSMs identified in the BV− and BV+ vaginal samples. In each of FIGS. 1A-1C, stars indicate comparisons that were significantly different between BV status by Mann-Whitney U Test (P<0.01).

FIGS. 2A-2E show biosynthesis of polyamines by D. micraerophilus and other BV-associated bacteria (BVAB). Dialister micraerophilus strains DSM19965 and DNF00843 were grown anaerobically in mono- and co-culture with Fannyhessea vaginae DSM15829, Peptoniphilus lacrimalis DNF00528, and Gardnerella vaginalis ATCC14018 in Brucella H&K media for 72 hrs, then pelleted and culture supernatants were collected. Cultures with D. micraerophilus DSM19965 are shown in dark gray and cultures with D. micraerophilus DNF00843 are shown in light gray. In FIG. 2A, a total polyamine concentration of culture supernatants is shown as measured by enzymatic assay. Bars represent standard error of measurements from three separate cultures. “ND” denotes measured polyamine concentrations below the limit of detection for the assay. Stars indicate statistically significant differences (Welch's T-test, P<0.05). Supernatants from three replicates of the D. micraerophilus mono-cultures and co-cultures with F. vaginae, in addition to cultures supplemented with 50 μg/mL L-ornithine, were pooled and analyzed by LC-MS/MS for putrescine (FIG. 2B), spermidine (FIG. 2C), and cadaverine (FIG. 2D). In each of FIGS. 2B-2D, the numbers above the bars indicate a measured peak area for the pooled samples. Spermine was not detected in any culture supernatants. FIG. 2E shows a schematic model of polyamine synthesis by D. micraerophilus aided by F. vaginae. “ADI” denotes arginine deiminase; “OCT” denotes “ornithine carbamoyltransferase”; “ODC” denotes “ornithine decarboxylase”; “SS” denotes spermidine synthase; and “LDC” denotes lysine decarboxylase.

FIGS. 3A-3G show evidence of bacterial fermentation elucidated by metaproteomics. FIG. 3A shows a summary of bacterial fermentation pathways observed in cervicovaginal lavage (CVL) samples. Genera associated with observed sample proteins are listed next to each enzymatic step. Different enzymatic pathways are grouped by numbers: (1) phosphoenolpyruvate carboxykinase, malate dehydrogenase, fumarate hydratase, and succinate dehydrogenase; (2) lactate dehydrogenase; (3) pyruvate: ferredoxin oxidoreductase; (4) pyruvate formate-lyase; (5) acetaldehyde dehydrogenase and alcohol dehydrogenase; (6) acetyl-CoA hydrolase; (7) phosphate acetyltransferase and acetate kinase. FIG. 3B shows a total mass of formate present in the supernatant of bacterial cultures grown anaerobically over 72 hrs. Culture media was supplemented with 0, 10, or 100 mmol of sodium pyruvate at the start of the incubation. Stars indicate a significant difference in the mass of formate compared to the 0 mmol control for the same isolate (Welch's T-test, P<0.05). “ND” denotes formic acid concentration of all replicates below limit of detection for the assay. Bars represent standard error of 3 replicates. FIG. 3C shows growth inhibition by a pyruvate formate-lyase (PFL)-inhibitor, sodium hypophosphite. Bacteria were cultured for 48 hrs in media with varying concentrations of hypophosphite, their growth in each condition was calculated by ΔOD600, then normalized to growth with 0 mM hypophosphite. Dotted line represents 100% growth relative to control. Stars indicate growth that was significantly lower than control (unpaired T-test, P<0.05). FIG. 3D shows inhibition of formic acid secretion by hypophosphite. Immediately following hypophosphite growth experiments, spent media of selected formate-producing bacteria were sampled and formate concentration was quantified.

FIG. 3E shows a density of Coriobacteriales bacterium DNF00809 (also named Umbribacter vaginalis) cultured in media containing 20 mM sodium formate or vehicle over 72 hrs. Bacterial density was measured by colony-forming unit (CFU) counting of the inoculum and samples taken every 24 hrs. Stars indicate a significant difference between treatments (unpaired T-test, P<0.05). FIG. 3F shows formate concentration in 20 mM formate DNF00809 (Umbribacter vaginalis) cultures and mock-inoculated control cultures, quantified by enzymatic assay. Formate concentration measured at time points subsequent to inoculation was normalized to the formate concentration of the cultures at the beginning of the experiment. Star indicates a significant difference in percent formate concentration between cultures (unpaired T-test P<0.05). In FIG. 3G, DNF00809 (Umbribacter vaginalis) consumption of formic acid produced by G. vaginalis is shown. G. vaginalis 315-A was cultured for 48 hrs and samples of media were taken pre- and post-incubation for formate concentration and pH quantification. G. vaginalis spent media was then filter-sterilized and inoculated with DNF00809 (Umbribacter vaginalis) or mock-inoculated, re-cultured for 120 hrs, then again sampled for formate concentration and pH quantification. In each of FIGS. 3D-3G, bars represent standard error of 3 separate cultures. Stars show significant differences between DNF00809 (Umbribacter vaginalis) cultures and mock-inoculated controls (unpaired T-test, P<0.05).

FIG. 4 is a bar graph showing concentration of human TGase3 in cervicovaginal fluid samples. Concentration of TGase3 in cervicovaginal fluid samples as measured by sandwich ELISA assay. BV status of samples was assessed by Nugent score. TGase3 was quantified in samples from 14 BV− and 12 BV+ participants. Black lines represent standard errors of the measurements. Dots represent concentration for individual samples. Star indicates a statistically significant difference (Welch's T-test, P<0.05).

FIG. 5 shows a simple schematic illustrating a use of formate analogs could disrupt diverse BVAB without harming commensal Lactobacilli.

### DETAILED DESCRIPTION

The present disclosure provides methods for treating bacterial vaginosis (BV) and reducing recurrence of BV through inhibition of formic acid metabolism in one or more BV-associated bacteria (BVAB). The disclosed methods are based at least in part on the discovery by the present inventors that formic acid metabolism is an important metabolic feature of multiple BVAB, and that inhibition of formic acid production and/or utilization selectively impairs BVAB while sparing beneficial Lactobacilli species. Accordingly, in certain embodiments, the present disclosure provides methods of treating BV or reducing recurrence of BV by administering an inhibitor of formic acid metabolism.

Reference will now be made in detail to embodiments, examples of which are illustrated in the accompanying drawings and examples below. In the following detailed description, numerous specific details are set forth to provide a thorough understanding of the present disclosure. However, it will be apparent to one of ordinary skill in the art that the present disclosure can be practiced without these specific details and in other instances, well-known methods, procedures, and elements have not been described in detail so as not to unnecessarily obscure aspects of the embodiments.

Unless otherwise defined in the present disclosure, all technical and scientific terms used herein have the meaning commonly understood by a person skilled in the art to which the disclosure belongs. The following references provide one of ordinary skill in the art with a general definition of many terms used herein: Singleton et al., Dictionary of Microbiology and Molecular Biology (3^{rd} ed. 2006); Cambridge Dictionary of Science and Technology (Walker ed. 1990); The Glossary of Genetics: Classical and Molecular, 5^{th} ed., Rieger et al. ed. 1991, Springer-Verlag, and Hale and Marham, The Harper Collins Dictionary of Biology (1991); Molecular Cloning: a Laboratory Manual 3^{rd} edition, Sambrook and Russell, ed. Cold Spring Harbor Laboratory Press 2001; Recombinant Antibodies for Immunotherapy, Melvyn Little, ed. Cambridge University Press 2009; “oligonucleotide Synthesis” (M. J. Gait, ed. 1984); “Animal Cell Culture” (RI Freshney, ed. 1987); “Methods in Enzymology” (Academic Press, Inc.); “Current Protocols in Molecular Biology” (FM Ausubel et al. eds. 1987, and periodic updates); ““PCR: The Polymerase Chain Reaction”, (Mullis et al., eds. 1994); “A Practical Guide to Molecular Cloning” (P. Bernard., 1988); and “Phage Display: A Laboratory Manual” (Barbas et al. eds. 2001). The contents of these references and other references containing standard protocols, widely known to and relied upon by those skilled in the art, including manufacturers' instructions, are hereby incorporated by reference as part of the presently disclosed subject matter. In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

All embodiments of any aspect of the disclosure can be used in combination, unless the context clearly dictates otherwise.

As used herein, the words “a” and “an,” when used in conjunction with the word “comprising” in the claims or specification, denote one or more, unless specifically noted.

As used in the specification and the appended claims, the term “and/or” when referring to two species, A and B, means at least one of A and B. As used in the specification and the appended claims, the term “and/or” when referring to greater than two species, such as A, B, and C, means at least one of A, B, or C, or at least one of any combination of A, B, or C (with each species in singular or multiple possibility).

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

As used herein, “about” or “comprising essentially of” mean within an acceptable error range for the particular value as determined by one of ordinary skill in the art, which will depend in part on how the value is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values are provided in the application and claims, unless otherwise stated, the meaning of “about” or “comprising essentially of” should be assumed to be within an acceptable error range for that particular value.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

As described herein, any concentration range, percentage range, ratio range or integer range is to be understood to include the value of any integer within the recited range and, when appropriate, fractions thereof (such as one tenth and one hundredth of an integer), unless otherwise indicated.

The term “subject”, as used herein, refers to an “animal” and in particular a “mammal” such as a non-primate (e.g., mice, rats, bovines, horses, household cats, tigers and other large cats, dogs, pigs, rabbits, goats, deer, sheep, ferrets, gerbils, guinea pigs, hamsters, bats, and birds (e.g., chickens, turkeys, and ducks)) or a primate (e.g., monkeys, baboons, chimpanzees, and human). The term may be used interchangeably with the term “patient” or “individual.” In some embodiments, the subject is a mammal, e.g., a human, diagnosed with a disease or disorder provided herein. In some embodiments, the subject is a mammal, e.g., a human, at risk of developing a disease or disorder provided herein.

As used herein, the term “biological sample” refers to any type of material of biological origin isolated from a subject, including, for example, DNA, RNA, protein, such as, for example, blood, plasma, serum, fecal matter, urine, semen, bone marrow, bile, spinal fluid, tears, saliva, muscle biopsy, organ tissue, or other material of biological origin known by those of ordinary skill in the art. Biological samples also include blood, serum, plasma, synovial fluid, bronchoalveolar lavage, spinal fluid, saliva, and the like including any bodily fluid.

As used herein, “treat” or “treating” means accomplishing one or more of the following: (a) reducing severity of symptoms of the infection in the subject; (b) limiting increase in symptoms in the subject; (c) increasing survival; (d) decreasing the duration of symptoms; (e) limiting or preventing development of symptoms; and (f) decreasing the need for hospitalization and/or the length of hospitalization for treating the infection. The term “treatment” refers to a pharmaceutical or other intervention regimen for accomplishing one or more of the above beneficial or desired results. Treating can be achieved with the eradication or amelioration of one or more of the physiological symptoms associated with the underlying disorder, such that an improvement is observed in the subject, notwithstanding that the subject may still be afflicted with the underlying disorder.

The term “effective amount” or “therapeutically effective amount”, as used herein, refers to an amount of an agent (e.g., a composition for marking a tissue as provided herein) that is sufficient to treat, diagnose, prevent, delay the onset of, reduce and/or ameliorate the severity and/or duration of a given condition, disorder, or disease and/or a symptom related thereto. The term also encompasses an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy.

The term “administer,” “administration,” or “administering,” as used herein, refers to the act of physically delivering a substance (e.g., a composition for treating bacterial vaginosis) to a subject, such as by oral, mucosal, topical, intradermal, parenteral, intravenous, intravitreal, intraarticular, subretinal, intramuscular, intrathecal delivery and/or any other method of physical delivery described herein or known in the art. The delivery can be systemic or to a specific tissue.

The term “prevent”, as used herein, refers to a pharmaceutical or other intervention regimen for reducing the likelihood of the onset (or recurrence) of a disease, disorder, condition, or associated symptom(s). Preventing includes delaying or eliminating the appearance of a disease or condition, delaying, or eliminating the onset of symptoms of a disease or condition, slowing, halting, or reversing the progression of a disease or condition, or any combination thereof.

As used herein, “reducing recurrence” includes decreasing the frequency, duration, severity, or likelihood of a condition, disorder, or disease and/or a symptom related thereto (e.g., bacterial vaginosis) returning after a prior treatment or remission.

The term “pharmaceutically acceptable excipient, carrier or diluent”, as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington, The Science and Practice of Pharmacy, 23rd edition, A. Adejare, ed., Academic Press, 2020).

The term “pharmaceutical composition” or “therapeutic composition”, as used here, refers to a composition capable of being administered to a subject for the treatment of a particular disease or disorder. Pharmaceutical or therapeutic compositions of some embodiments of the present disclosure can be manufactured by suitable processes, e.g., by means of mixing, dissolving, granulating, dragee-making, levigating, emulsifying, encapsulating, entrapping or lyophilizing processes.

As used herein, pharmaceutical or therapeutic compositions for use in accordance with some embodiments of the disclosure thus can be formulated in a conventional manner using one or more physiologically acceptable carriers comprising excipients and auxiliaries, which facilitate processing of the active agents into preparations which can be used pharmaceutically. Proper formulation is dependent upon the route of administration chosen and is well known to the skilled artisan.

As used herein, pharmaceutical or therapeutic compositions suitable for use in context of some embodiments of the present disclosure include compositions wherein the active agents are contained in an amount effective to achieve the intended purpose.

Determination of a therapeutically effective amount or an effective amount is well within the capability of those skilled in the art, especially in light of the detailed disclosure provided herein. For any preparation used in the methods of the present disclosure, the therapeutically effective amount or dose can be estimated initially from in vitro and cell culture assays. For example, a dose can be formulated using animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in humans. The dosage can vary depending upon the dosage form employed and the route of administration utilized. The exact formulation, route of administration and dosage can be chosen by the individual physician in view of the patient's condition.

As described herein, depending on the severity and responsiveness of the condition to be treated, dosing can be of a single or a plurality of administrations, with course of treatment lasting from several days to several weeks or until cure is affected or diminution of the disease state is achieved. The amount of a composition to be administered will, of course, be dependent on the subject being treated, the severity of the affliction, the manner of administration, the judgment of the prescribing physician, and the like.

As used herein, the term “cell” includes, for example, various cells derived from individual animals, subcultured cells, primary cultured cells, cell lines, recombinant cells, and microbial cells.

The term “host,” as used herein, refers to an animal, such as a mammal (e.g., a human).

The term “host cell,” as used herein, refers to a particular subject cell into which an exogenous nucleic acid molecule can be introduced and the progeny or potential progeny of such a cell. Progeny of such a cell may not be identical to the parent cell comprising the nucleic acid molecule due to mutations or environmental influences that can occur in succeeding generations or integration of the nucleic acid molecule into the host cell genome.

As used herein, the term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue, and the like.

As used herein, the term “gene” means a nucleic acid molecule comprising a nucleotide sequence encoding the amino acids of a protein, or its complementary strand. The “gene” is meant to include, for example, a polynucleotide, and oligonucleotide, DNA, mRNA, cDNA, and cRNA comprising a nucleotide sequence encoding the amino acids of a protein or a nucleotide sequence complementary thereto. Such a gene is a single-stranded, double-stranded, or triple or more stranded nucleotide. The “gene” is also meant to include an association of DNA and RNA strands, a mixture of ribonucleotides (RNAs) and deoxyribnucleotides (DNAs) on one nucleotide strand, and a double-stranded or triple or more stranded nucleotide comprising such a nucleotide strand. For instance, examples of the “PFL gene” of the present invention can include DNA, mRNA, cDNA, and cRNA comprising a nucleotide sequence encoding the amino acid sequence of the PFL protein.

The term “polynucleotide” or “nucleic acid”, as used herein, refers to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides can comprise modified nucleotides or bases, and/or their analogs, or any substrate that can be incorporated into a polymer by DNA or RNA polymerase or by a synthetic reaction. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction.

As used herein, the term “nucleotide” has the same meaning as “nucleic acid” and “nucleic acid molecule,” and is also meant to include, for example, DNA, RNA, probe, oligonucleotide, polynucleotide, and primer. Such a nucleotide is a single-stranded, double-stranded, or triple or more stranded nucleotide. The “nucleotide” is also meant to include an association of DNA and RNA strands, a mixture of ribonucleotides (RNAs) and deoxyribonucleotides (DNAs) on one nucleotide strand, and an association of two strands or three or more strands comprising such a nucleotide strand.

The term “isolated nucleic acid,” as used herein, refers to a nucleic acid, for example, an RNA, DNA, or a mixed nucleic acids, substantially separated from other genomic DNA sequences as well as proteins or complexes such as ribosomes and polymerases that naturally accompany a native sequence, or culture medium when produced by recombinant techniques, or chemical precursors or other chemicals when chemically synthesized.

The term “amino acid residue” or “amino acid” includes reference to an amino acid that is incorporated into a protein, polypeptide, or peptide. The term “amino acid” refers to a molecule containing both an amino group and a carboxyl group. In certain embodiments, the amino acid is an alpha-amino acid. In certain embodiments, the amino acid is a natural amino acid. In certain embodiments, the amino acid is a non-natural amino acid. There are many known non-natural amino acids, any of which may be included in the peptides of the present invention. See for example, S. Hunt, The Non-Protein Amino Acids: In Chemistry and Biochemistry of the Amino Acids, edited by G. C. Barrett, Chapman and Hall, 1985.

The amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The terms “amino acid,” “amino acid residue,” “amino acid sequence,” or polypeptide sequence include naturally occurring amino acids (including L and D isosteriomers) and, unless otherwise limited, also include known analogs of the twenty common natural amino acids that can function in a similar manner, such as, e.g., selenocysteine, pyrrolysine, N-formylmethionine, gamma-carboxyglutamate, hydroxyprolinehypusine, pyroglutamic acid, and selenomethionine (see e.g., Nagata K et al., Bioinformatics 30:1681-9 (2014)).

The terms “polypeptide” and “peptide” and “protein”, as used herein, refer to polymers of amino acids of any length. The polymer may be linear or branched, it may comprise modified amino acids, and it may be interrupted by non-amino acids. The terms, as used herein, refer to proteins, miniproteins, polypeptides, and peptides of any size, structure, or function. The terms may also refer to dimers or oligomers of peptides. The terms, as used herein, may include stapled, unstapled, stitched, and unstitched polypeptides. Typically, a peptide will be at least three amino acids long. A peptide may refer to an individual peptide or a collection of peptides. Inventive peptides preferably can comprise only natural amino acids or also non-natural amino acids (i.e., compounds that do not occur in nature but that can be incorporated into a peptide chain) and/or amino acid analogs as are known in the art May alternatively be employed. One or more of the amino acids in a peptide may be modified, for example, by the addition of a chemical entity such as a carbohydrate group, a hydroxyl group, a phosphate group, a farnesyl group, an isofamesyl group, palmitoyl, geranylgeranyl, lauryl, a fatty acid group, a linker for conjugation, functionalization, or other modification. A peptide may be just a fragment of a naturally occurring protein or peptide. The terms also encompass an amino acid polymer that has been modified naturally or by intervention; for example, disulfide bond formation, glycosylation, lipidation, acetylation, phosphorylation, or any other manipulation or modification. A peptide, polypeptide, or protein may be naturally occurring, recombinant, or synthetic, or any combination thereof. Also included within the definition are, for example, polypeptides containing one or more analogs of an amino acid, including but not limited to, unnatural amino acids, as well as other modifications known in the art.

The term “binds” or “binding,” as used herein, refers to a covalent or non-covalent interaction between molecules (e.g., forming a complex by interactions). Exemplary non-covalent interactions include hydrogen bonds, ionic bonds, hydrophobic interactions, and/or van der Waals interactions. As used herein, the term “specifically binds” refers to binding of an antibody or an antigen binding fragment thereof to an antigen with a dissociation constant (K_{D})≤10⁻⁷M. The term “K_{D}” is intended to refer to the dissociation equilibrium constant of a particular antibody-antigen interaction. The ratio of dissociation rate (k_{off}) to association rate (kₒₙ) of an antibody to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. Specific binding can be measured, for example, by determining binding of a molecule compared to binding of a control molecule, which generally is a molecule of similar structure that does not have binding activity.

The term “binding affinity,” as used herein, refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen, or a molecule and its ligand). The affinity of a binding molecule X for its binding partner Y can generally be represented by the dissociation constant (K_{D}). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

As used herein, the terms “binding ligand” and “affinity reagent” refer to any molecule having an ability to bind to a specific target molecule (e.g., an antigen of interest, a target antigen, a small molecule, and/or a specific chemical group) with a specific affinity (i.e., detectable over background). Binding ligand molecules and affinity reagent molecules are known and have been characterized for useful antigens and are encompassed by the present application without limitation. Exemplary and non-limiting categories of binding ligands and/or affinity reagents that can be used in the context of the present disclosure include antibodies, and antigen-binding fragments, and derivatives thereof.

As used herein, the term “small molecule” refers to a low molecular weight organic or inorganic compound, typically having a molecular weight of less than about 1,500 Da, that is capable of modulating a biological process. Small molecules may include, for example, naturally occurring compounds, synthetic compounds, or semi-synthetic derivatives, and can exist as salts, esters, solvates, hydrates, stereoisomers, mixtures thereof, and the like.

While each of the elements of the present disclosure is described herein as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

The referenced patents, patent applications, and scientific literature referred to herein are hereby incorporated by reference in their entirety as if each individual publication, patent or patent application were specifically and individually indicated to be incorporated by reference.

In one aspect, the present disclosure provides a method of treating BV or reducing recurrence of BV in a subject by administering to the subject a therapeutically effective amount of an inhibitor of formic acid metabolism in one or more BVAB. In one example of the method, the inhibitor reduces growth of the one or more BVAB. The inhibitor can also reduce metabolic activity of the one or more BVAB. In another example, the inhibitor reduces both growth and metabolic activity.

Administration of the inhibitor results in reduced symptoms of BV, including but not limited to reduced bacterial burden, reduced malodor, reduced abnormal discharge, restoration of normal vaginal pH, a reduction in inflammation, or combinations thereof.

Without wishing to be bound by theory, formic acid may function as an important fermentation product and exchanged metabolite within polymicrobial communities associated with BV. Interruption of formic acid production, uptake, or utilization may therefore disrupt cooperative interactions (e.g., metabolic interactions) that contribute to persistence or recurrence of BV.

The therapeutically effective amount of the inhibitor is an amount effective to inhibit formic acid production by one or more BVAB. The therapeutically effective amount of the inhibitor can also be effective to inhibit formic acid consumption by one or more BVAB. In some examples, the therapeutically effective amount of the inhibitor inhibits both production and consumption of formic acid of one or more BVAB and/or within a BV− associated microbial community. Furthermore, inhibition of formic acid production can reduce availability of formate to neighboring organisms. In one example, inhibition of formic acid consumption suppresses organisms that rely on formate as an energy source or growth-promoting metabolite.

The inhibitor of the present disclosure can be an inhibitor of pyruvate formate lyase (PFL). PFL catalyzes conversion of pyruvate to acetyl-CoA and formate under anaerobic conditions and may be utilized by multiple BVAB. Inhibition of PFL can reduce formate secretion, decrease fermentative metabolism, and/or suppress growth of formate-producing BVAB.

The inhibitor of the present disclosure can also be an inhibitor of formate dehydrogenase (FDH). FDH catalyzes the reversible oxidation of formate to carbon dioxide, thereby generating energy used for anaerobic respiration and metabolic pathways. FDH inhibition can also reduce utilization of formate as an energy source by formate-consuming BVAB. In some examples, inhibition of FDH disrupts syntrophic interactions between formate-producing and formate-consuming organisms present in BV. In addition, inhibition of FDH can suppress organisms that benefit from cross-feeding of formate produced by neighboring bacteria.

In some embodiments, the inhibitor of the present disclosure can comprise a formate analog or a formic acid analog that competes with, mimics, or otherwise interferes with enzymes or transport systems involved in formate metabolism. As used herein, a “formate analog” and a “formic acid analog” refers to a compound that structurally resembles formate and formic acid, respectively. Such formate analogs or formic acid analogs can interact with one or more enzymes, transporters, or pathways involved in formate metabolism. For example, a formate analog can bind to an enzyme active site and compete with formate. These formate analogs and formic acid analogs can also be transported into a cell via a cellular formate transport system, thereby disrupting intracellular metabolic processes. Suitable analogs may include salts, esters, prodrugs, derivatives, solvates, hydrates, stereoisomers, or combinations thereof.

As described herein, the inhibitor of the present disclosure can comprise a small molecule that interferes with formate metabolism. In some examples, the aforementioned formate analog or formic acid analog is a small molecule. The small molecule inhibitor can, for example, inhibit production, secretion, uptake, transport, or utilization of formic acid or formate. For instance, the small molecule inhibitor can inhibit pyruvate conversion to formate and/or downstream oxidation of formate. In some examples, the small molecule inhibits the enzymatic activity of PFL and/or FDH.

Small molecule inhibitors may act through direct or indirect interactions with their biological targets. For example, a small molecule inhibitor as described herein can function as a ligand that binds to an enzyme involved in formate metabolism (e.g., PFL). Such binding may occur at an active site, catalytic pocket, substrate binding site, cofactor binding site, or allosteric site, which results in alteration of a conformation, stability, and/or catalytic activity of the enzyme. The small molecule inhibitors can act as competitive inhibitors by mimicking a natural substrate, such as formate or pyruvate, and occupying the active site of the enzyme or a catalytic intermediate. Alternatively, the small molecule inhibitors can act as a non-competitive or uncompetitive inhibitor by binding to a site distinct from the substrate binding site that results in modulation of the enzyme function. Furthermore, the small molecule inhibitors can interact with cofactors (e.g., redox cofactors) or catalytic radicals required for an enzyme activity. In one example, the small molecule inhibitors can interfere with the radical-based catalysis and/or disrupt activation of PFL. In another example, the small molecule inhibitors can perturb the metal centers or redox cofactors associated with FDH.

Ligand binding of the small molecule exhibits a measurable affinity for the target, such as a dissociation constant (K_{D}), inhibition constant (K_{I}), or half-maximal inhibitory concentration (IC50) within a therapeutically relevant range.

In some embodiments, the inhibitor comprises hypophosphite or a pharmaceutically acceptable salt thereof. In one example, the inhibitor comprises sodium hypophosphite. Hypophosphite inhibits PFL activity in and reduces formate production by one or more BVAB. In some examples, hypophosphite functions as a substrate analog or transition state mimic that binds to PFL and inhibits conversion of pyruvate to formate, thereby reducing formate production and suppressing growth and/or metabolic activity of formate-producing BVAB.

In one example, the inhibitor comprising hypophosphite or a pharmaceutically acceptable salt thereof is administered at a concentration effective to reduce formate production while preserving beneficial vaginal bacteria (e.g., one or more Lactobacillus species). The selectively arises from, for example, differences among the bacteria in enzyme presence, structure, and/or metabolic reliance on formate pathways.

In some embodiments, the one or more BVAB comprise Gardnerella vaginalis, Gardnerella piotii, Gardnerella leopoldii, Gardnerella swidsinskii, Fannyhessea vaginae, Amygdalobacter indicium, Amygdalobacter nucleatus, Megasphaera lornae, Megasphaera vaginalis, Megasphaera curtisii, Megasphaera mulieris, Prevotella bivia, Sneathia vaginalis, Umbribacter vaginalis, or a combination thereof.

In some embodiments, the methods disclosed herein are directed to treatment of polymicrobial BV communities comprising two or more of the foregoing BVAB.

In some embodiments, the inhibitor of the present disclosure selectively prevents or reduces growth, viability, colonization, metabolic activity, or a combination thereof, of one or more BVAB relative to one or more Lactobacillus species. In one example, the inhibitor does not impact or does not substantially impact growth of the one or more Lactobacillus species. In certain embodiments, the one or more Lactobacillus species comprise Lactobacillus crispatus, Lactobacillus jensenii, Lactobacillus gasseri, Lactobacillus iners, or a combination thereof. As mentioned, selectivity can be a result of differences among the bacteria in enzyme presence, structure, and/or metabolic reliance on formate pathways.

In some embodiments, the inhibitor of the present disclosure is formulated as a topical composition for intravaginal application. In some embodiments, suitable forms of the topical composition include gels, creams, ointments, foams, suppositories, tablets, capsules, films, rings (e.g., intravaginal rings), inserts, douches, irrigations, sprays, powders, or liquid suspensions. The topical composition can further comprise one or more pharmaceutically acceptable carriers, buffers, preservatives, stabilizers, viscosity modifiers, mucoadhesive agents, controlled release excipients, or a combination thereof.

In one example, the inhibitor of the present disclosure is administered intravaginally. Local administration may provide locally elevated concentrations effective to suppress BVAB while limiting systemic exposure.

The inhibitor of the present disclosure can be administered in combination with an antibiotic for treating BV. Suitable antibiotics include but not limited to metronidazole, clindamycin, secnidazole, tinidazole, or combinations thereof. A combination therapy of an antibiotic and the inhibitor can improve cure rate, reduces recurrence, lowers antibiotic dose, promotes recolonization by one or more Lactobacillus species, or a combination thereof. In one example, the inhibitor and antibiotic are administered concurrently. In another example, the inhibitor is administered sequentially with the antibiotic. In yet another example, the inhibitor is administered after completion of an antibiotic treatment for BV. Post-antibiotic administration of the inhibitor can reduce or prevent recurrence of BV by suppressing reestablishment of one of more BVAB and/or BV-associated microbial communities.

In some examples, the inhibitor is administered to a subject having BV for treating the BV. In other examples, the inhibitor can also be administered prophylactically to a subject at risk of BV recurrence. Subjects at risk of BV recurrence may include subjects with a prior history of recurrent BV, altered vaginal microbiota, recent antibiotic exposure, hormonal changes, sexual practices associated with recurrence risk, or other predisposing factors. Prophylactic administration of the inhibitor can be periodic, intermittent, cyclic, or continuous for a selected duration.

Administration of the inhibitor of the present disclosure can promote restoration of a vaginal microbiota dominated by one or more Lactobacillus species. In one example, administration of the inhibitor decreases an abundance of BVAB. In another example, administration of the inhibitor increases an abundance of one or more Lactobacillus species.

Administration of the inhibitor can also reduce vaginal malodor associated with BV. Without wishing to be bound by theory, disruption of formate-dependent metabolism may reduce abundance or activity of organisms that participate in generation of volatile amines or other odor causing metabolites.

Administration of the inhibitor can further lower vaginal pH. In one example, administration of the inhibitor lowers vaginal pH through promoting reestablishment of lactic acid-producing bacteria (e.g., Lactobacillus species). In another example, administration of the inhibitor lowers vaginal pH by reducing the abundance and/or metabolic activity of BVAB that raises vaginal pH by, for instance, synthesizing polyamines, secreting ammonia, and/or consuming lactic acid.

Administration of the inhibitor can improve one or more diagnostic markers of BV, including but not limited to Nugent score, Amsel criteria, molecular microbiome measurements, reduction in BV-associated taxa, increase in Lactobacillus abundance, reduction in odor, normalization of discharge, reduction in recurrence over time, patient-reported symptom improvement, or a combination hereof.

While illustrative embodiments have been illustrated and described, it will be appreciated that various changes can be made therein without departing from the spirit and scope of the present disclosure.

The embodiments of the disclosure are further illustrated by the following examples, which are only illustrative and are not intended to limit the definition and scope of the disclosure in any way.

### Example

### Syntrophic Bacterial and Host-Microbe Interactions in Bacterial Vaginosis

An optimal vaginal microbiota is dominated by Lactobacillus spp., often with a single species constituting greater than 50% of the community. These commensal bacteria help exclude pathogens from the vagina and upper reproductive tract by fermenting vaginal glycogen into lactic acid and secreting antimicrobial peptides. In bacterial vaginosis (BV), diverse anaerobic and facultative bacteria increase in abundance compared to commensal Lactobacilli. These BV-associated bacteria (BVAB) perform functions that can be detrimental to host health, such as degrading the cervicovaginal mucus barrier, attacking host cells with cytotoxins, and secreting malodorous amines.

BV is a highly prevalent condition, affecting approximately 29% of reproductive-aged women in the U.S. as assessed by Gram stain of vaginal fluid. Women from disadvantaged socioeconomic groups are at higher risk of BV, and having a BV bacterial community is associated with increased risk of adverse sequelae such as pelvic inflammatory disease, preterm birth, and acquisition of sexually transmitted infections. Although antibiotic therapy is effective in treating BV, up to 60% of patients experience a recurrence within one year. Alternative and supplemental therapies to antibiotics have only modestly improved rates of cure or recurrence, highlighting the need for a better understanding of BV pathogenesis.

BV is a polymicrobial condition of the vaginal microbiota, and no single organism is sufficient to cause disease. BV is notable for the presence of heterogeneous bacterial communities, further complicating the study of this condition. Interbacterial interactions are thought to be important in maintaining this diverse community structure. Previous in vitro studies have found examples of interbacterial relationships, such as Prevotella bivia feeding ammonia to Gardnerella and Peptostreptococcus, and Gardnerella vaginalis promoting the growth of Fannyhessea vaginae in a biofilm culture system. Known interactions between BVAB only involve a small number of taxa, however, and other interactions likely exist that help stabilize the BV community. Host-bacterial interactions also likely play a role in BV. Bacterial catabolism of host-derived amino acids into foul-smelling amines such as cadaverine and putrescine is thought to contribute to BV malodor. Similarly, bacterial breakdown of host mucus likely contributes to reduced mucosal barrier capacity and the characteristic thin vaginal discharge. But less is known about the host response to BV and how it can ameliorate or exacerbate the condition.

Untargeted metaproteomics is a powerful tool to study biological processes in complex cellular communities such as those found in the human vagina. By identifying proteins present in the vagina and associating them with the host or specific bacterial taxa, functions which influence the composition of the microbiota can be identified. In the present example, metaproteomic data were analyzed from cervicovaginal lavage (CVL) samples from 20 women with BV and 9 without BV to uncover in vivo host and bacterial processes. A high-level, statistical approach was used to determine what host and bacterial proteins are differentially abundant based on BV status. A low-level analysis was also applied, examining proteins with low spectral counts but biologically important functions. This combination of high- and low-level analysis has revealed broad functional differences between the BV and non-BV proteome, which may contribute to the condition, in addition to novel capabilities and interactions in the BV microbiota that could represent promising targets for disruption by future therapeutics.

### Materials and Methods

Study population and sample collection. The study and sample collection methods were approved by the Institutional Review Board at the Fred Hutchinson Cancer Center and the University of Washington. All study participants provided written informed consent. Participants were recruited from the Seattle & King County Sexual Health Clinic, University of Washington Infectious Diseases/Virology Research Clinic, Hall Health Student Clinic at the University of Washington, University of Washington OB-GYN Clinic, and Harborview Vaginitis Clinic. A random cross-sectional case-control set of 21 samples from women with BV and 9 samples from women without BV were selected for proteomics and metagenomics. As there is greater bacterial heterogeneity in BV, a 2:1 case was used to control ratio for sample collection. One sample could not be analyzed, leaving 29 total. Cervicovaginal lavage (CVL) samples were collected for molecular characterization, Gram-staining, pH, saline microscopy, and potassium hydroxide preparation. CVL was collected by instilling 10 mL sterile saline into the vagina using a needleless syringe, and walls of the vagina were washed to remove any adherent cells. After approximately 1 minute, the lavage fluid was aspirated and stored at −80 °C. BV was diagnosed using Amsel clinical criteria and confirmed by Gram-stain using the Nugent and Hillier method. BV diagnosis was concordant by both Nugent score and Amsel criteria for all 29 participants whose samples were analyzed by mass spectrometry. CVL was used for proteomic analyses. A separate set of 60 CVL samples that was used for formic acid quantitation was collected as part of the same parent study and Nugent score was used to determine their BV status.

Cervicovaginal fluid (CVF) samples used for confirmation of transglutaminase 3 (TGase3) levels were self-collected by participants for a longitudinal study of BV and HSV-2 infection. Participants inserted a menstrual cup (SoftCup®) into the vagina for 5 to 10 minutes. The menstrual cup was placed in a 50 ml conical tube and kept in the participant's freezer and subsequently stored at −80 °C until processing. BV status for participants was assessed by Nugent score.

Swab preparation and DNA extraction from vaginal swab samples. Vaginal swabs collected from study participants were frozen at −80 °C until processing. Swabs were prepared for extraction by placing swab tips in a tube with 500 μL filtered (100K MWCO) 0.9% saline. Swabs tips in the tubes were mixed by vortex for 1-2 min and then removed. Vaginal fluid from each swab tip was centrifuged at 14,000 rpm for 10 min at 4 °C to pellet cells. Genomic DNA was extracted from pellets using the BiOstic® Bacteremia DNA Isolation Kit (Qiagen, Germantown, MD, USA). DNA was eluted in 150 μL buffer. DNA extraction controls (blank swab without contact with human mucosa) were included for every 15 samples to assess contamination from extraction reagents or collection swabs. DNA was stored at −80 °C until analysis.

Broad-range PCR and sequencing for microbiota characterization. Total bacterial DNA concentrations (16S rRNA gene copies) were measured using a qPCR assay targeting the V3-V4 region of the 16S rRNA gene. Samples were evaluated for the presence of PCR inhibitors using a qPCR assay targeting a segment of exogenously added jellyfish DNA and inhibition was defined as a delay in the threshold cycle (Cₜ) of >2 cycles compared to no-template controls. Relative abundances of bacterial sequence reads were measured using broad-range PCR targeting the V3-V4 region of the 16S rRNA gene and sequencing on the MiSeq™ System (Illumina, San Diego, CA). Raw sequence reads were demultiplexed using the MiSeq™ System's onboard software. Demultiplexed reads were processed using barcodecop v0.4.1 (Hoffman NG. barcodecop. 2019) to enforce barcode quality using default settings and to ensure exact barcode matches to the forward and reverse reads. The DADA2 package version 1.6.0 was used for quality filtering, read trimming, error correction and dereplication, paired-end assembly and chimera removal resulting in a list of unique sequence variants. Sequence variants were classified using the phylogenetic placement tool pplacer and a curated set of urogenital bacteria. Sequence reads are available from the NCBI Short Read Archive (Accession number PRJNA881379).

Metagenomics library preparation and sequencing. Genomic DNA (gDNA) from vaginal samples was quantified via Qubit™ fluorometer and Quant-iT™ dsDNA Assay Kit, high sensitivity (Life Technologies-Invitrogen, Carlsbad, CA, USA). Sequencing controls included a bacterial mock community (ATCC MSA-1003), and a bacterial isolate DNF00720 Fannyhessea vaginae. Nucleotide sequencing libraries were prepared from 250 μg gDNA with a quarter reaction workflow using the Nextera® XT Library Prep Kit (Illumina, San Diego, CA, USA). Libraries were pooled by volume and post-amplification cleanup was performed with 0.8× Agencourt AMPure® XP beads (Beckman Coulter, Indianapolis, IN, USA). The library pool size distribution was validated using the Agilent® High Sensitivity D5000 ScreenTape run on an Agilent 4200 TapeStation® (Agilent Technologies, Inc., Santa Clara, CA, USA). Additional library QC and cluster optimization was performed using a Qubit™ 2.0 Fluorometer (Life Technologies-Invitrogen, Carlsbad, CA, USA). Nucleotide sequencing was performed on a NovaSeq® 6000 S1-300 flowcell (Illumina, San Diego, CA, USA). Image analysis and base calling were performed on-board the NovaSeq® 6000 with Real Time Analysis v3.4.4 software. Generation of Fastq files was performed with Illumina's bcl2fastq 2.20 Conversion software. The geneshot workflow was used to process Fastq files. This workflow fed data into metaSPAdes for assembly and Prokka for annotation of bacterial genes. Taxonomic annotation was performed using MetaPhlAn2.

Proteomic sample preparation. Cervicovaginal lavage (CVL) samples were reduced, denatured, and digested to peptides for mass spectrometry analysis. First, 105 mg of solid urea was added to 250 μL of CVL to yield a final concentration of 7 M. Dithiothreitol was then added to a final concentration of 5 mM and the sample was incubated for 30 minutes at 60 °C with gentle shaking (300 rpm) in a thermomixer. Following incubation, 2.25 mL of 50 mM ammonium bicarbonate was added to each tube. Trypsin (USB) was resuspended to 1 μg/μL in acetic acid, and 5 μL was added to each sample. Samples were incubated at 37 °C with gentle shaking (300 rpm) overnight. Digested peptide samples were centrifuged for 5 minutes at 12,000×g to pellet any remaining solid debris and the supernatant was subjected to solid phase extraction (SPE). SPE cartridges (Phenomenex; Strata C18-T (55 μm, 140 Å), 100 mg/1 mL) were loaded into the vacuum manifold and samples were processed according to the manufacturer's recommendation. Briefly, cartridges were conditioned with 1 mL methanol and washed with 1 mL 0.1% trifluoroacetic acid (TFA) in water. The sample was added to the cartridge, followed by a wash with 1 mL 5% acetonitrile/95% 0.1% TFA in water. Finally, the sample was eluted with 1 mL 80% acetonitrile and 20% 0.1% TFA in water. Samples were concentrated to near dryness using a vacuum centrifuge (SpeedVac™) and resuspended in 30 μL 0.1% TFA water. A BCA assay (Pierce) was performed to determine peptide concentration. Samples were diluted to 0.1 μg/μL and stored at −80 °C until MS analysis.

LC-MS/MS analysis. A dual pumping Ultra-performance liquid chromatography system (nanoAcquity™, Waters, Milford, MA) was configured for on-line trapping of a 5 μL injection at 5 μL/min with reverse-flow elution onto the analytical column at 300 nL/min. Columns were packed in-house using 360 μm o.d. fused silica (Polymicro Technologies Inc., Phoenix, AZ) with 2-mm sol-gel frits for media retention and contained Jupiter C18 media (Phenomenex, Torrence, CA) in 5 μm particle size for the trapping column (150 μm i.d.×4 cm long), with 3 μm particle size for the analytical column (75 μm i.d.×70 cm long). Mobile phases consisted of (A) 0.1% formic acid in water and (B) 0.1% formic acid in acetonitrile with the following gradient profile (min, % B): 0, 1; 2, 8; 20, 12; 75, 30; 97, 45; 100, 95; 110, 95; 115, 1; 150, 1. MS analysis was performed using a mass spectrometer (Velos Orbitrap®, Thermo Scientific, San Jose, CA) outfitted with a custom electrospray ionization (ESI) interface. Electrospray emitters were custom made by chemically etching 150 μm o.d.×20 μm i.d. fused silica. The heated capillary temperature and spray voltage were 350 °C and 2.3 kV, respectively. Data was acquired for 100 minutes after a 15-minute delay from when the gradient started. Spectra (AGC 1×10⁶) were collected from 400 to 2000 m/z at a resolution of 60 k followed by data-dependent ion trap MS/MS (collision energy 35%, AGC 1×10⁴) of the 10 most abundant ions. A dynamic exclusion time of 45 seconds was used to discriminate against previously analyzed ions using a −0.55- to 1.55-Da mass window. Each sample was analyzed in two separate replicates.

Database construction for identification of peptide sequences. A sample-specific database was built to separately analyze each sample as described in Lee et al. (2023). Briefly, all databases included human protein sequences from Swiss-Prot (release 2019_02), 16 common contaminants (detailed below), translated bacterial genes identified in the sample by shotgun metagenomic sequencing, and RefSeq protein sequences for bacterial taxa present in the sample at >0.1% abundance according to 16S rRNA gene sequencing (RefSeq release 99, downloaded Jun. 19, 2020), except Amygdalobacter indicium and Amygdalobacter nucleatus for which there were no available genomes at the time of analysis. All bacterial protein sequences were normalized before being incorporated into databases with the SpeciesSeqPrepper.py program. Gardnerella species were delineated using the same groups described in Vaneechoutte, et al. (2019). Recently published Gardnerella genomes were submitted to the DSMZ genome-to-genome distance calculator and a cutoff of 70% similarity was used to determine Gardnerella species.

Database searching. Approximately half of bacterial peptides identified in searches of the first replicates were not identified in the second replicates and vice-versa, so both replicates for each sample were combined for further proteomic analysis. Peptide identification was performed with MS-GF+ (v2019.01.22). Isotope error range was −1 to 2, maximum modifications per peptide was set to 3, and peptides were only considered if they were at least partially tryptic. The Nextflow® workflow manager (v19.07.0) was used to parallelize and automate the data analysis pipeline.

A two-step database search method was used to maximize data from each sample. Briefly, after an initial database search was completed, every protein linked to a peptide-spectrum match, regardless of statistical significance, was recorded and their sequences were included in a subset protein database. A second search was performed using these subset databases, so that only proteins identified in the first search were compared against the sample spectra, and the results of these second searches were used for downstream analysis. A decoy database was created and searched concurrently to calculate false discovery rate (FDR) and q values.

Proteomic data analysis. Search results were analyzed in Jupyter®Lab (v1.1.4) using Python® (v3.6.4). False discovery rate was limited by only including peptide-spectrum matches (PSMs) with a Q-value <0.01. To identify sample proteins, peptide spectra were classified in a hierarchical manner. If the spectrum matched any decoy proteins, it was flagged as a decoy identification and discarded. Next, if the spectrum matched any contaminant proteins, it was flagged as a contaminant identification and similarly discarded. Otherwise, if the spectrum matched any human proteins in the database, it was categorized preferentially as human. Finally, the spectrum was characterized as bacterial if it only matched bacterial proteins. Peptides were included in further analysis if MS-GF+ assigned the PSM a spectral probability value <1E-15 or the peptide was one of two unique peptides that matched to the same protein.

Differential protein abundance analysis. To calculate the relative abundance of human proteins, the total number of PSMs attributed to a protein in a sample was divided by the total number of human PSMs in that sample. Due to their heterogeneity, bacterial proteins were grouped functionally by annotating them with the eggNOG-Mapper web server (v1.0.3). For bacterial PSMs that matched multiple proteins, only the first protein match was queried. Proteins were then grouped based on the “Preferred_name” field assigned to each by eggNOG-Mapper, and relative abundance of each group in a sample determined by dividing the number of PSMs matching that group by the total number of bacterial PSMs in that sample. Protein relative abundances were then log transformed (base 2) for statistical analysis. Statistical tests were performed using the scipy (v1.3.1) “stats” package. Mann-Whitney U tests were used to assess differences in relative abundance of specific proteins across samples.

Taxonomic analysis of qPCR, DNA sequencing, and proteomic data. Taxonomic analysis was performed at the genus level to better compare data between techniques. For qPCR data, the number of reads for each genus was summed, then divided by the combined number of reads for all qPCR assays performed on the sample to calculate relative abundance. For shotgun metagenomic sequencing data, “estimated_number_of_reads_from_the_clade” value for each genus in the MetaPhlAn2 output was divided by the total number of reads in the sample to calculate relative abundance of each genus. Genera with a relative abundance of <0.5% were excluded to minimize false positive identifications. Taxonomic assignment of peptides was performed using the taxonomic information attached to protein sequences in each database. When analyzing taxonomic makeup of samples, spectra that matched multiple genera were excluded so ambiguous hits would not skew the analysis. When analyzing identified bacterial proteins for biological relevance, all taxa matching sample spectra were noted so functions of minority organisms would not be excluded.

Contaminant protein sequences. Proteins such as keratins and trypsins are commonly introduced into samples by processing procedures and from the researchers themselves. Therefore, the following contaminant proteins were included in all metaproteomic databases as a minimal set of peptides that should be excluded from downstream analysis while simultaneously attempting to preserve true identifications. These proteins were: Sus scrofa trypsin precursor (sp|P00761|TRYP_PIG), Promega trypsin artifact 1 (Trypa1), Promega trypsin artifact 2 (Trypa2), Promega trypsin artifact 3 (Trypa3), Promega trypsin artifact 4 (Trypa4), Promega trypsin artifact 5 (Trypa5), Trypsin artifact 6 (Trypa6), Bos taurus trypsinogen (sp|P00760|TRYP_BOVIN), Bos taurus chymotrypsinogen A (CTRA_BOVIN), Bos taurus chymotrisinogen B (CTRB_BOVIN), Homo sapiens serum albumin precursor (sp|P02768|ALBU_HUMAN), Bos taurus serum albumin precursor (sp|P02769|ALBU_BOVIN), Homo sapiens keratin type II cytoskeletal 1 (K2C1_HUMAN), Homo sapiens keratin type II cytoskeletal 2 (K22E_HUMAN), Homo sapiens keratin type I cytoskeletal 9 (K1C9_HUMAN), and Homo sapiens keratin type I cytoskeletal 10 (K1C10_HUMAN).

Protein homolog searching with BLASTp. The stand-alone Protein-Protein BLAST program (v2.12.0+) was used to identify protein homologs in bacterial genomes. Query sequences were searched against a database that contained all the bacterial proteins used to build the sequence databases for peptide spectrum identification. Default parameters were used for the searches. A bacterial species was determined to possess a homolog of the query protein if at least one isolate encoded a protein that produced a match with an E-value<0.0001. The following proteins were used as queries: pyruvate formate-lyase sp|P09373|PFLB_ECOLI from E. coli, formate dehydrogenase sp|P07658|FDHF_ECOLI from E. coli, anaerobic ribonucleoside-triphosphate reductase sp|P28903|NRDD_ECOLI from E. coli, glutamine synthetase tr|S4HEE6|S4HEE6_GARVA from G. vaginalis, glutamate dehydrogenase sp|P00370|DHE4_ECOLI from E. coli, glutamate transporter tr|S4HL05|S4HL05_GARVA from G. vaginalis, TMAO reductase sp|P33225|TORA_ECOLI from E. coli, betaine reductase sp|069407|GRDH_PEPAC from Peptoclostridium acidaminophilum, choline trimethylamine-lyase sp|Q30W70|CUTC_OLEA2 from Oleide sulfovibrio alaskensis, carnitine monooxygenase sp|P0ABR7|CNTA_ECOLI from E. coli, arginine deiminase sp|P13981|ARCA_PSEAE from Pseudomonas aeruginosa, ornithine carbamoyltransferase sp|P08308|OTCC_PSEAE from P. aeruginosa, ornithine decarboxylase sp|P24169|DCOS_ECOLI from E. coli, agmatinase sp|P60651|SPEB_ECOLI from E. coli, putrescine carbamoyltransferase sp|Q837U7|PTC_ENTFA from E. faecalis, acetylpolyamine amidohydrolase 2 sp|Q9I6H0|APAH2_PSEAE from P. aeruginosa, putrescine-pyruvate aminotransferase sp|Q916J2|SPUC_PSEAE from P. aeruginosa.

Bacterial proteins identified in the CVL samples were searched for homologs of proteins of interest as described above, using a database populated with all bacterial proteins identified in the samples.

Screening bacterial isolates for formic acid fermentation, utilization, and depletion. The bacterial isolates used in these experiments were Lactobacillus crispatus ATCC33197, Lactobacillus iners DSM13335, Lactobacillus gasseri DSM20243, Lactobacillus jensenii DSM20557, Gardnerella vaginalis ATCC14018, Gardnerella vaginalis UPII 315-A, Gardnerella piotii JCP8066, Gardnerella leopoldii CCUG72425, Gardnerella swidsinskii CCUG72429, Fannyhessea vaginae DSM15829, Amygdalobacter indicium UPII 610-J, Amygdalobacter nucleatus KA00274, Megasphaera lornae UPII 199-6, Megasphaera vaginalis BV3C16-1, Prevotella bivia DSM20514, Sneathia vaginalis DSM16631, and Coriobacteriales bacterium DNF00809 (also named Umbribacter vaginalis). The lactobacilli, G. vaginalis, and G. piotii isolates were grown on NYCIII 1.5% agar plates. The G. leopoldii, G. swidsinskii, F. vaginae, Amygdalobacter, Megasphaera, Prevotella, and DNF00809 isolates were grown on Brucella H&K agar plates (Hardy Diagnostics, Santa Maria CA). The S. vaginalis isolate was recovered from frozen growth directly in pBHI+5% fetal calf serum (FCS) liquid media. The lactobacilli, Gardnerella, and F. vaginae isolates were cultured in NYCIII liquid media. The Megasphaera and P. bivia isolates were cultured in PYG-mod-YG liquid media. The Amygdalobacter isolates were cultured in CRM-YG+3% FCS liquid media. The Sneathia and DNF00809 isolates were cultured in WC+3% FCS liquid media. At each subculture, Gram stains were performed to verify culture purity. All culturing took place in an AS-580 anaerobic chamber (Anaerobe Systems, Morgan Hill, CA) in a gas mixture of 5% CO₂, 5% H₂, and 90% N₂ at 37 °C.

To test bacteria for formic acid fermentation, isolates were recovered from frozen stocks on solid media for 24 hrs-72 hrs depending on the rate of growth of the isolate. When sufficient growth had been achieved, the bacteria were inoculated into 2 mL liquid culture and allowed to grow for another 24 hrs. Experimental cultures were then prepared by aliquoting 1 mL liquid media into sterile 1.5 mL tubes (Axygen, Union City, CA) and adding 0 μL, 10 μL, or 100 μL of filter-sterilized 1M sodium pyruvate (Sigma-Aldrich, St. Louis, MO) so that the total mass of supplemental pyruvate in each tube was 0 mmol, 10 mmol, or 100 mmol, 100 μL of bacterial culture was then inoculated into the tubes. Three replicates were made for each isolate in each pyruvate treatment. Uninoculated control tubes were also made for each media type with all three quantities of added pyruvate. The experimental and control tubes were then incubated for 48 hrs. Following this incubation, the tubes were removed from the anaerobic chamber, centrifuged at 10,000×g for 10 min, then 900 μL of supernatant was collected and stored at −20 °C until the concentration of formic acid could be assayed. Formic acid concentrations were quantified with a formic acid assay kit (Megazyme®, Bray, Ireland). 10 μL of thawed supernatant was tested according to the assay's microplate procedure in clear-bottom 96 well plates (Corning Inc., Corning, NY) on a plate reader (BioTek® Epoch™ 2, Agilent, Santa Clara, CA). Bacterial culture supernatant formic acid concentrations were calculated against a standard curve, then matched media blanks were subtracted to determine the concentration of formic acid in the supernatant. This concentration was multiplied by the total volume of the culture to determine the total mmol of formate secreted by the bacteria. Welch's T-tests were performed to compare formate in the supernatants of cultures with 10 mmol or 100 mmol of added sodium pyruvate to the 0 mmol controls. Differences were considered significant if P<0.05.

To test bacteria for the ability to deplete formate from their media, isolates were recovered on agar media, then sub-cultured into 2 mL of liquid media and cultured for 24 hrs. Experimental cultures were made by mixing 800 μL liquid media, 100 μL filter-sterilized 500 mM sodium formate (Sigma-Aldrich, St. Louis, MO), and 100 μL of bacterial culture in a sterile 1.5 mL tube (QIAGEN, Hilden, Germany). Three replicate tubes were made for each isolate. Additionally, a heat-inactivated control was prepared for each isolate by centrifuging 900 μL of bacterial culture at 10,000×g for 10 min to pellet the cells, removing the supernatant and resuspending the cells in 900 μL fresh media to remove any formate they had secreted while growing, then placing the tubes in a water bath at 70 °C for 45 min. The control tubes were allowed to cool, then 100 μL 500 mM sodium formate was added. The control and experimental tubes were incubated for 48 hrs, then removed from the anaerobic chamber, centrifuged at 10,000×g for 10 min, then 800 μL of supernatant was removed and stored at −20 °C until the remaining formate could be assayed. Thawed supernatants were diluted 1:2, then the concentration of formate was measured using the assay kit as described above. The concentration of formate remaining in the supernatants of the experimental cultures was normalized against their heat-inactivated controls.

To test bacteria for the ability to utilize formate as a carbon and energy source, isolates were recovered on agar media, then sub-cultured into 2 mL of liquid media and cultured for 24 hrs. No-Glucose media stock solutions were made by preparing liquid media without glucose and using 90% the volume of H₂O. Experimental media was then made by adding filter-sterilized 500 mM glucose, 500 mM sodium formate, or water at 1:10 volume to the No-Glucose media stock solution. 180 μL of experimental media was aliquoted into wells on a 96 well plate (Corning Inc., Corning, NY), the plate was sealed with a sealing film (Axygen UltraClear Sealing Film, Axygen, Union City, CA), then transferred to a glove box (PLAS Labs Model 830 glove box, PLAS Labs, Lansing, MI) and OD600 was recorded every 30 min for 72 hrs using a plate reader (BioTek® Epoch™ 2 plate reader, Agilent, Santa Clara, CA) at 37 °C in 10% CO₂, 90% N₂.

Monitoring bacterial growth with supplemental sodium pyruvate. Gardnerella piotii JCP8066, Gardnerella swidsinskii CCUG72429, and Amygdalobacter nucleatus KA00274 were recovered from frozen stocks on solid media as described above. Culture purity was confirmed by Gram stain, then the bacteria were inoculated into 2 mL liquid media and cultured anoxically at 37 °C for 24 hrs. To achieve the same concentrations of pyruvate as were used in the above experiments measuring formic acid production, experimental media was prepared by mixing 1M filter-sterilized sodium pyruvate to the isolate's preferred media at a volume ratio of 0:1, 1:100, or 1:10. 200 μL of experimental media was then aliquoted into wells of a 96 well plate (Corning Inc., Corning, NY), and 20 μL of bacterial culture were inoculated into the media. Four replicate wells were prepared for each combination of isolates and media treatments. The plate was sealed with a sealing film (Axygen® Ultra Clear Sealing Film, Axygen, Union City, CA), then transferred to a glove box (PLAS Labs Model 830 glove box, PLAS Labs, Lansing, MI) and OD600 was recorded every 30 min for 72 hrs using a plate reader (BioTek® Epoch™ 2 plate reader, Agilent, Santa Clara, CA) at 37 °C in 10% CO₂, 90% N₂. Bacterial growth was calculated by subtracting the lowest OD600 measurement in the well from the highest the isolate achieved during the 72 hrs incubation. Relative growth of bacteria in media supplemented with additional formate was calculated by comparing them to a paired well of the same isolate with no added pyruvate.

Measuring effect of hypophosphite on bacterial growth and formic acid production. Isolates used in these experiments were the same as those tested for formic acid production, with the addition of L. crispatus MV-1A-US and G. vaginalis ATCC14019. Bacteria were recovered from frozen stocks on agar plates then subcultured into liquid media as described previously. A stock solution of 5 M sodium hypophosphite (Thermo Fisher Scientific, Waltham, MA) was prepared and filter-sterilized by passing it through a 0.1 μm PVDF syringe filter (EMD Millipore, Darmstadt, Germany). The stock solution was then serially diluted to 250 mM, 100 mM, and 10 mM. 20 μL of sodium hypophosphite solutions or H₂O were added to wells of a sterile 96-well polystyrene plate (Corning Inc., Corning, NY). 160 μL of media was added to the wells, then 20 μL of bacterial culture was inoculated into wells. 3 replicate cultures were prepared for each isolate in each hypophosphite condition. The plate was sealed with film (Axygen® Ultra Clear Sealing Film, Axygen, Union City, CA), then placed in a plate reader (BioTek® Epoch™ 2 plate reader, Agilent®, Santa Clara, CA) inside an anaerobic chamber (Concept, Baker, Sanford, ME) and incubated at 37 °C for 48 hrs, shaking for 30 sec every 30 min then measuring OD600 of each well. ΔOD600 was calculated for each timepoint by subtracting the initial OD600 reading from the reading at the timepoint. Percent growth was calculated by normalizing measurements to the average ΔOD600 for the 3 cultures at 0 mM hypophosphite. Significant differences were determined by unpaired T-test (P<0.05).

For bacterial isolates where formic acid inhibition was measured, 100 μL of media were sampled immediately after the final OD600 reading and stored at −20 °C. Samples were later thawed and formate concentration was quantified with a formic acid assay kit (Megazyme, Bray, Ireland). Significant differences in formic acid concentration of spent media were determined by unpaired T-test (P<0.05).

DNF00809 testing for formic acid utilization. Coriobacteriales bacterium DNF00809 (also named Umbribacter vaginalis) was recovered from frozen stocks on Brucella plates then subcultured into WC-3% FCS liquid media. Experimental media was made by preparing WC-3% FCS media without any added glucose, then adding filter-sterilized sodium formate (Sigma-Aldrich, St. Louis, MO) solution to a final concentration of 20 mM formate, or water to serve as a vehicle control. 100 μL of DNF00809 culture was inoculated into 900 μL of 20 mM formate or vehicle control media and mixed well. 100 μL sterile media was inoculated into 900 μL 20 mM formate media to serve as a negative control for formate depletion. 3 replicate cultures were prepared for each condition. 100 μL of each culture was immediately sampled for formate quantification and stored at −20 °C, then cultures were incubated at 37 °C under anoxic conditions. Every 24 hrs, another 100 μL of each culture was sampled for formic acid quantification and CFU counting. CFUs of input and timepoint samples were quantified by serially diluting to 1E1, 1E2, 1E3, and 1E4, then spotting five 10 μL drops onto Brucella plates. CFU counting plates were incubated at 37 °C for 48 hrs, then visible colonies were counted on a single dilution to calculate CFUs/mL at the timepoint. Once the final sample had been collected, all samples were thawed and analyzed for formate concentrations using a formic acid assay kit (Megazyme, Bray, Ireland). Formate concentrations were normalized to the 0 hr concentration in the same sample to calculate percent formate concentration. Significant differences in CFUs/mL between 20 mM formate versus vehicle media and percent formate concentration in DNF00809 versus mock-inoculated controls were calculated by unpaired T-test (P<0.05).

DNF00809 consumption of formate produced by bacteria was tested by culturing G. vaginalis 315-A in 4 mL PYG-mod-YG+3% FCS media under anoxic conditions for 48 hrs. 3 replicate cultures were prepared. 100 μL samples of each culture were taken pre- and post-incubation and stored at −20 °C. G. vaginalis cultures were then pooled, centrifuged at 10,000×g for 10 min, then supernatants were filter-sterilized using a 0.1 μm PVDF syringe filter (EMD Millipore, Darmstadt, Germany). 600 μL spent media were then aliquoted into 1.5 mL tubes (QIAGEN, Hilden, Germany) and 100 μL DNF00809 culture or sterile WC+3% FCS media were inoculated in. 3 replicate tubes were prepared for both experimental and control cultures. The tubes were incubated anoxically at 37 °C for 120 hrs, then 100 μL were sampled from each. Presence of live DNF00809 bacteria were confirmed by plating 10 μL of each culture onto Brucella plates and allowing them to grow under anoxic conditions at 37 °C for 48 hrs. All samples were thawed and their pH measured by spotting 30 μL onto pH strips (MColorpHast™ pH 4.0-7.0 pH strips, MilliporeSigma, Burlington, MA) and formate concentrations were measured using a formic acid assay kit (Megazyme, Bray, Ireland). Significant differences between DNF00809 cultures and controls were determined by unpaired T-test (P<0.05).

Measurement of formic acid concentrations in CVL. Formic acid concentrations were quantified with a formic acid assay kit (Megazyme, Bray, Ireland). CVL samples were thawed, briefly mixed by vortex to homogenize, then 20 μL of sample and standards were tested according to the assay's microplate procedure in clear-bottom 96 well plates (Corning Inc., Corning, NY) on a plate reader (BioTek® Epoch™ 2 plate reader, Agilent, Santa Clara, CA). Three blanks were tested alongside each batch of samples, and the limit of detection for the assay was defined as a blank-adjusted change in OD340 greater than the mean for the three blanks. This equaled a minimum detectable formate concentration of 0.128 mM. Sample formate concentrations were then calculated against a standard curve.

Quantification of polyamine concentrations in bacterial culture supernatants. The bacterial isolates used in these experiments were Dialister micraerophilus DSM19965, Dialister micraerophilus DNF00843, Fannyhessea vaginae DSM15829, Peptoniphilus lacrimalis DNF00528, and Gardnerella vaginalis ATCC14018. The bacteria were first grown in pure culture by recovering them from frozen stocks on Brucella H&K agar plates (Hardy Diagnostics®, Santa Maria, CA), followed by two subcultures into 2 mL Brucella H&K media. At each step, bacteria were grown in an anaerobic chamber (AS-580, Anaerobe Systems, Morgan Hill, CA) in a gas mixture of 5% CO₂, 5% H₂, and 90% N₂ at 37 °C for 72 hrs and purity of the cultures was confirmed between each subculture by Gram stain. These pure cultures of actively growing bacteria were then used to inoculate the experimental cultures. For mono-cultures, 200 μL of bacterial culture was inoculated into 1.8 mL Brucella H&K media. For co-cultures, 100 μL of each bacterial isolate was inoculated into 1.8 mL Brucella H&K media. A mono-culture of each isolate was grown, and a co-culture was set up so both D. micraerophilus isolates grew in co-culture with the F. vaginae, P. lacrimalis, and G. vaginalis isolates. Three replicates of each culture were made in separate tubes. Experimental cultures were grown anoxically at 37 °C for 72 hrs. Following this incubation, cultures were briefly mixed by vortex to break up biofilms that had formed on the bottom of culture tubes, then 1 mL of each culture was removed and centrifuged at 10,000×g for 10 minutes to pellet cells. The supernatant was removed and stored at −20 °C until polyamine quantification was performed.

Polyamine quantification was performed with a polyamine assay kit (Fluorometric Total Polyamine Assay Kit, Sigma-Aldrich, St. Louis, MO). To clean up samples prior to analysis, culture supernatants were thawed and 4 μL Sample Clean-Up Mix was added to 200 μL of supernatant. The mixture was incubated at room temperature for 30 minutes, then transferred to centrifugal concentrators (10 kDa Cut-Off Corning Spin-X® UF Concentrators, Corning Inc., Corning, NY). Cleaned-up samples were diluted 1:100, 1:500, and 1:1000 in ultrapure distilled water (Invitrogen, Waltham, MA), then these diluted samples were analyzed according to kit specifications. Fluorescence of samples was read on a fluorescence microplate reader (Biotek® FL×800 fluorescence microplate reader, Agilent, Santa Clara, CA). Differences in polyamine concentration between samples was calculated by performing Welch's T-test to compare. Differences were considered significant if P<0.05.

Targeted LC-MS for quantification of polyamines. In addition to the D. micraerophilus cultures described above, additional cultures with exogenous ornithine were grown. L-ornithine (Sigma-Aldrich, St. Louis, MO) was dissolved in water to a concentration of 500 μg/mL, then filter-sterilized by passing it through a 0.2 μm sterile nylon filter (Thermo Fisher Scientific, Waltham, MA). 100 μL of pure culture of D. micraerophilus DSM19965 or D. micraerophilus DNF00843, and 200 μL of 500 μg/mL L-ornithine were then added to 1.7 mL Brucella H&K liquid media, bringing the final concentration of L-ornithine in the culture to 50 μg/mL. Three replicates of these cultures for both D. micraerophilus isolates were set up in separate tubes. The bacteria were then cultured anoxically at 37 °C for 72 hrs, then culture supernatants were collected as described above. Prior to preparation for liquid chromatography with mass spectrometry (LC-MS) analysis, culture supernatants were thawed, 50 μL of each were pooled, briefly mixed using a vortex mixer, then stored at −20 °C until sample preparation was performed.

Four polyamines (putrescine, cadaverine, spermidine, and spermine) for targeted LC-MS analysis were extracted using a protein precipitation method. Samples were first homogenized in 200 μL purified deionized water at 4 °C, and then 800 μL of methanol were added. Samples were mixed using a vortex mixer, stored for 30 min at −20 °C, sonicated in an ice bath for 10 min, centrifuged for 15 min at 18,000×g and 4 °C, and then 600 μL of supernatant was collected from each sample. Lastly, recovered supernatants were dried on a vacuum centrifuge (SpeedVac™, Eppendorf, Enfield, CT) and reconstituted in 0.5 mL of LC-matching solvent for LC-MS acquisition.

Relative quantitation of the four polyamines was performed on a LC-MS system composed of two UPLC pumps (Shimadzu, Kyoto, Japan), a temperature-controlled auto-sampler (PAL® HTC-xt temperature-controlled auto-sampler, CTC Analytics, Zwingen, Switzerland), and a triple quadrupole MS equipped with ESI ionization source (6500+ Triple Quadrupole MS equipped with ESI ionization source, AB SCIEX, Toronto, Canada). The four polyamines were separated on a UPLC amide column (XBridge™ BEH Amide column, Waters, Milford, MA) running in hydrophilic interaction liquid chromatography (HILIC) mode. Solvent A was 10 mM ammonium acetate in 95% water, 2% MeOH, 3% acetonitrile, 0.2% acetic acid. Solvent B was 10 mM ammonium acetate in 5% water, 2% MeOH, 93% acetonitrile, 0.2% acetic acid. The total chromatography time was 18 min, followed by 18 min for reconditioning the column. The retention times (RTs) for putrescine, cadaverine, spermidine and spermine were 9.03, 8.90, 10.28 and 8.51 min, respectively. The separated molecules were ionized in a positive ionization mode (ESI+) and MS data acquisition was performed in multiple-reaction-monitoring (MRM) mode. The MRM transitions (precursor/product ion pairs) for putrescine, cadaverine, spermidine and spermine were 89⁺/72⁺, 103⁺/86⁺, 146⁺/72⁺ and 203⁺/129⁺, respectively. The LC-MS platform was controlled using Analyst® 1.7.2 software (Sciex, Redwod City, CA). Measured MS peaks were integrated using MultiQuant™ 3.0.3 software (Sciex, Redwod City, CA).

Transglutaminase 3 quantification. 10× protease inhibitor solution was made by dissolving 2 tablets of cOmplete™, Mini, EDTA-free protease inhibitor cocktail (Roche, Basel, Switzerland) in 1 mL of 100 mM HEPES buffer at pH 8.5 (Sigma-Aldrich, St. Louis, MO). Cervicovaginal fluid (CVF) samples were thawed, briefly mixed using a vortex mixer to homogenize them, then 150 μL of CVF was added to 150 μL of 1× protease inhibitor solution and stored at −80 °C. Concentration of transglutaminase 3 in the samples was quantified with a human transglutaminase 3 enzyme-linked immunosorbent assay kit (Invitrogen, Waltham, MA). Samples were serially diluted 1:20, 1:200, 1:1000, 1:2000, and 1:10000 in 1× assay diluent D, treated according to kit specifications, then absorbance of the samples was measured at 450 nm and concentration calculated against a standard curve. The scipy (v1.3.1) “stats” package was used to perform a Welch's T-test to compare the average concentration of transglutaminase 3 in CVF samples from participants with BV to those without BV.

### Results

BV is associated with proteins reflecting more diversified bacterial metabolism. Cervicovaginal lavage (CVL) samples from 20 women with BV (BV+) and 9 women without BV (BV−) were analyzed by species-specific quantitative PCR (qPCR), broad-range 16S rRNA gene sequencing, metagenomic sequencing, and liquid chromatography with tandem mass spectrometry (LC-MS/MS). The taxonomic makeup of the samples was generally similar between these 4 methods. One exception was sample BV+_6, which was dominated by Candidatus Lachnocurva, but metagenomic sequencing indicated this sample mostly contained Megasphaera, likely reflecting an absence of Candidatus Lachnocurva nucleotide sequences in the metagenomic database. Some genera were over- or under-represented in the metaproteomic data. In samples containing Gardnerella, this genus had on average a 2.2× higher abundance in the peptide data compared to 16S rRNA gene sequencing data. Conversely, Sneathia had a 4.7× lower abundance on average in metaproteomic data compared to 16S rRNA gene sequencing. It is unclear whether these differences reflect differential metabolic activity by these taxa, or greater availability of Gardnerella protein sequences in our database.

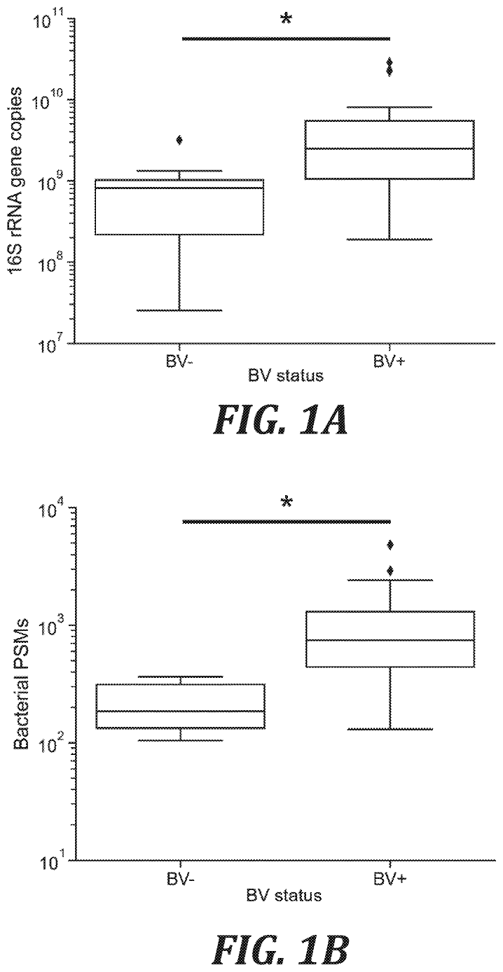

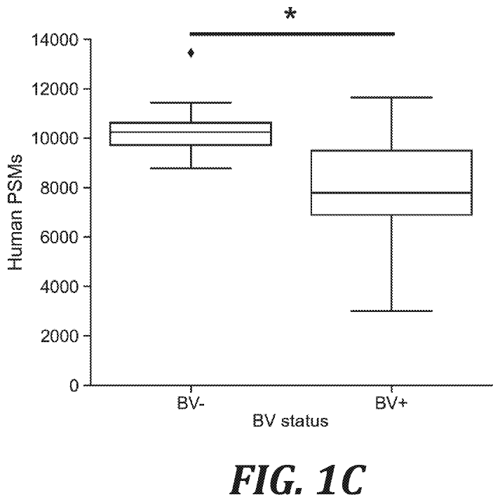

In total, 1,068 unique human proteins and 1,418 unique bacterial proteins were identified. To elucidate functional differences in the vaginal proteome during BV, proteins that were significantly differentially abundant based on BV status were first determined. To account for a lower number of human peptide-spectrum matches (PSMs) in BV+ samples, the abundance of each human protein was separately normalized to the total number of human spectra identified in each sample, and vice versa for bacterial proteins. Bacterial proteins involved in general metabolism such as RNA polymerase, ATP synthase, and ribosomal proteins had significantly higher relative abundances in samples from BV+ participants (Table 1). Bacterial load as measured by total 16S rRNA gene copies per sample was also significantly higher in BV+ samples (FIG. 1A). On average, 5× as many bacterial PSMs was identified in the BV+ samples compared to BV−(Mann-Whitney U test, P<0.001) (FIG. 1B). Significantly fewer human PSMs was identified in BV+ samples (Mann-Whitney U test, P<0.001) (FIG. 1C). Thus, bacterial proteins were a larger share of the overall proteome in BV+ samples.

Glycolysis enzymes were some of the most identified proteins in the data of the present example, but both human and bacterial glycolysis enzymes were less abundant in BV+ samples (Table 1). Conversely, bacterial glycosidases, which can release metabolizable sugars from vaginal glycogen, were more abundant in BV+ samples (Table 1). These results indicate that, although there is increased competition for carbohydrates in BV, carbohydrates make up a smaller overall percentage of bacterial metabolism in BV. This interpretation is supported by the higher abundance of bacterial proteases and amino acid catabolic enzymes in BV+ samples (Table 1).

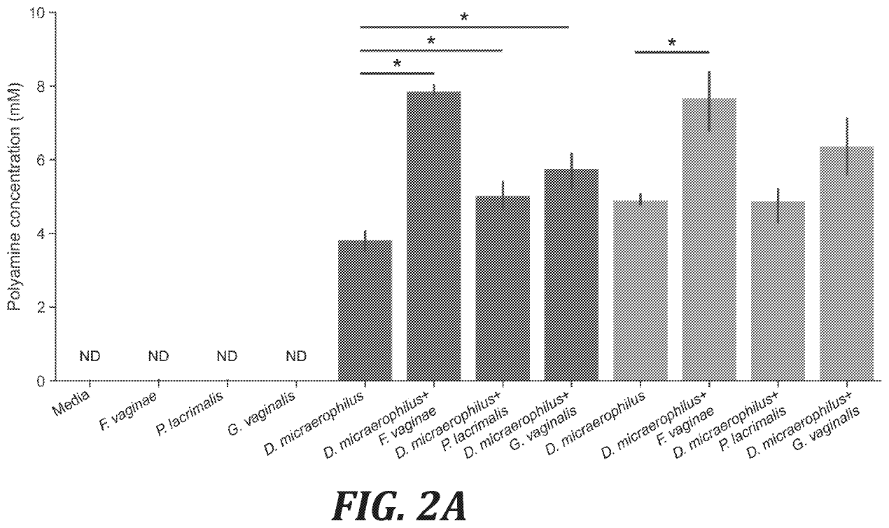

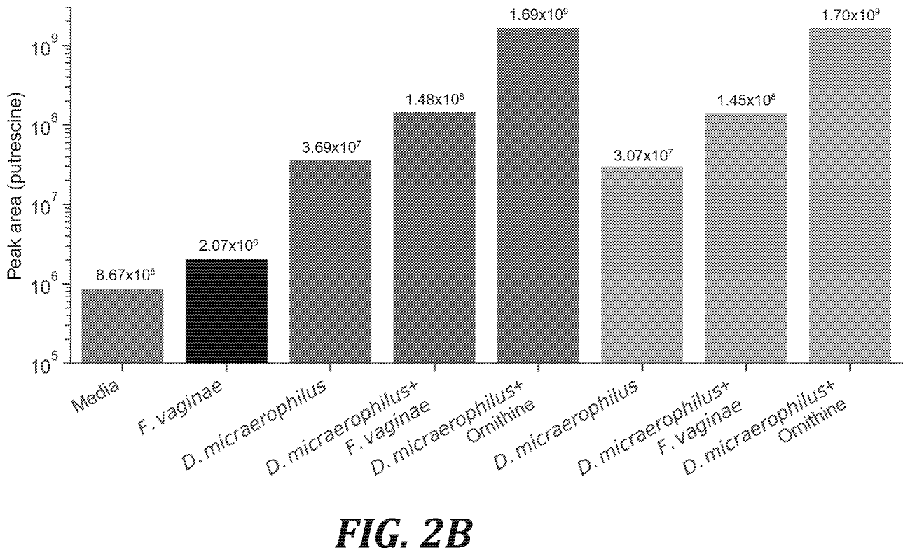

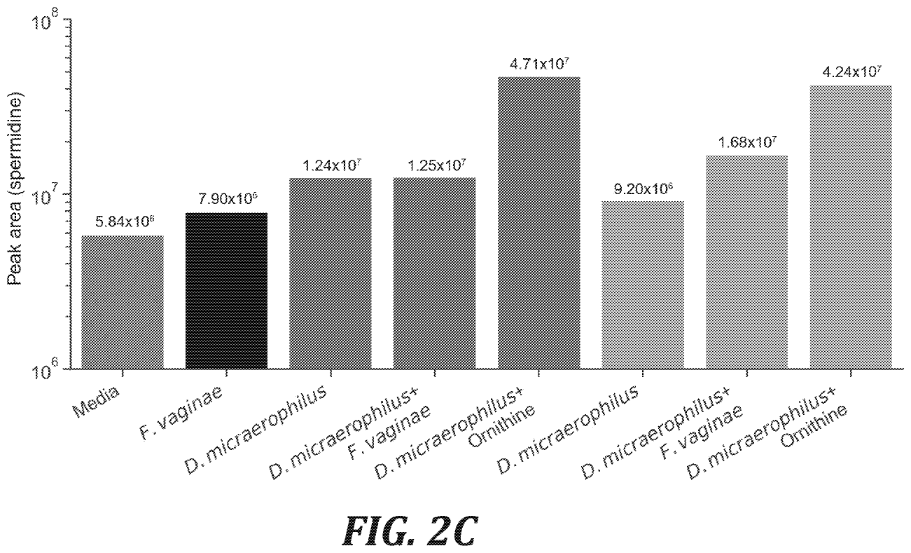

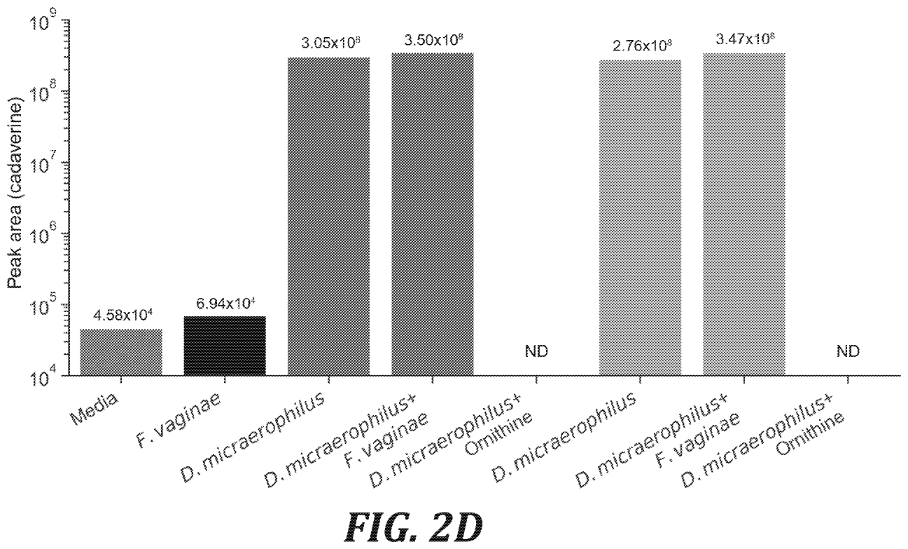

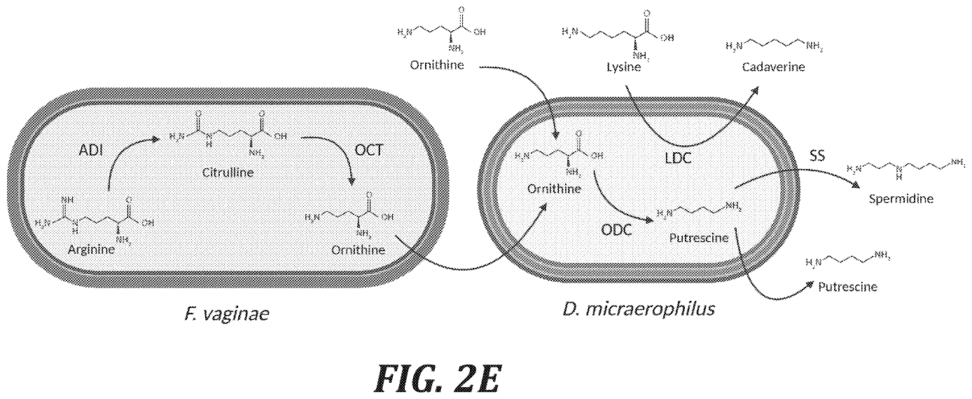

Dialister micraerophilus cooperates with other BVAB to increase polyamine synthesis. The concentrations of unpleasant-smelling compounds such as trimethylamine, cadaverine, and putrescine are commonly elevated in BV, which likely contributes to the malodor associated with the condition. Among the bacterial proteins in the present data, a complete pathway to convert arginine into putrescine was identified: arginine deiminase (ADI), ornithine carbamoyltransferase (OCT), and ornithine decarboxylase (ODC), but different enzymes were associated with different species. Spectra matching ADI and OCT were identified from both Fannyhessea vaginae and Sneathia vaginalis (Table 2). Spectra matching ODC were associated exclusively with Dialister micraerophilus. Genomic analysis supported these proteomic data; F. vaginae and S. vaginalis genomes lacked ODC whereas D. micraerophilus possessed homologs of ODC but not ADI or OCT (Table 3). None of these organisms possessed homologs of other putrescine-synthesizing enzymes including agmatinase, putrescine carbamoyltransferase, acetylpolyamine amidyhydrolase 2, or putrescine-pyruvate aminotransferase. Based on these data, the present inventors hypothesized that F. vaginae and D. micraerophilus can form a syntrophic metabolic relationship to synthesize putrescine. To test this hypothesis, two isolates of D. micraerophilus were grown in mono-culture and in co-culture with F. vaginae, then quantified the total polyamines present in their culture supernatants using an enzymatic assay. Because G. vaginalis does not encode putative ornithine-producing enzymes whereas Peptoniphilus lacrimalis encodes a homolog of the ornithine-producing enzyme arginase (Table 3), mono- and co-cultures with these organisms were also tested. F. vaginae, G. vaginalis, and P. lacrimalis did not synthesize detectable concentrations of polyamines in mono-culture, but both isolates of D. micraerophilus produced high concentrations of polyamines (FIG. 2A). In line with the present inventors' hypothesis, both D. micraerophilus isolates produced significantly higher concentrations of polyamines in co-culture with F. vaginae (Welch's T-test, P<0.05). Although D. micraerophilus DSM19965 produced a significantly higher concentration of polyamines in co-culture with both P. lacrimalis and G. vaginalis as expected, differences in polyamine concentrations for the D. micraerophilus DNF00843 isolate in co-cultures with these other bacteria were not statistically significant suggesting strain level differences in bacterial metabolism.

To determine the precise polyamines D. micraerophilus synthesized in mono- and co-cultures with F. vaginae, targeted metabolomics was performed by liquid chromatography with mass spectrometry (LC-MS) for putrescine, spermidine, spermine, and cadaverine. Supernatants from all three replicates of the D. micraerophilus mono- and co-cultures with F. vaginae were pooled for analysis. Cultures of both D. micraerophilus isolates in media supplemented with 50 μg/mL L-ornithine were also analyzed to determine whether this precursor alone could increase putrescine biosynthesis. Spermine was not detected in any of the supernatants, but putrescine was found in all eight pooled culture supernatants (FIG. 2B). Both D. micraerophilus strains made putrescine in mono-culture, possibly because the blood in their rich media supplied some ornithine. The concentration of putrescine was 4 times higher in co-cultures of D. micraerophilus with F. vaginae than mono-cultures, and much higher when the culture media was supplemented with ornithine, consistent with the present inventors' hypothesis. Spermidine only increased when exogenous ornithine was added to the culture media (FIG. 2C). Although both D. micraerophilus isolates synthesized cadaverine in mono- and co-culture with F. vaginae, cadaverine was undetectable in cultures supplemented with ornithine (FIG. 2D). These results suggest that on its own, D. micraerophilus can scavenge lysine as a precursor for cadaverine biosynthesis and can make putrescine from ornithine when the latter compound is present in its environment. Other BVAB like F. vaginae might supply additional ornithine to increase putrescine biosynthesis (FIG. 2E). Biosynthesis of cadaverine and putrescine are likely co-regulated in D. micraerophilus because an excess of the putrescine precursor ornithine led to abrogation of cadaverine production.

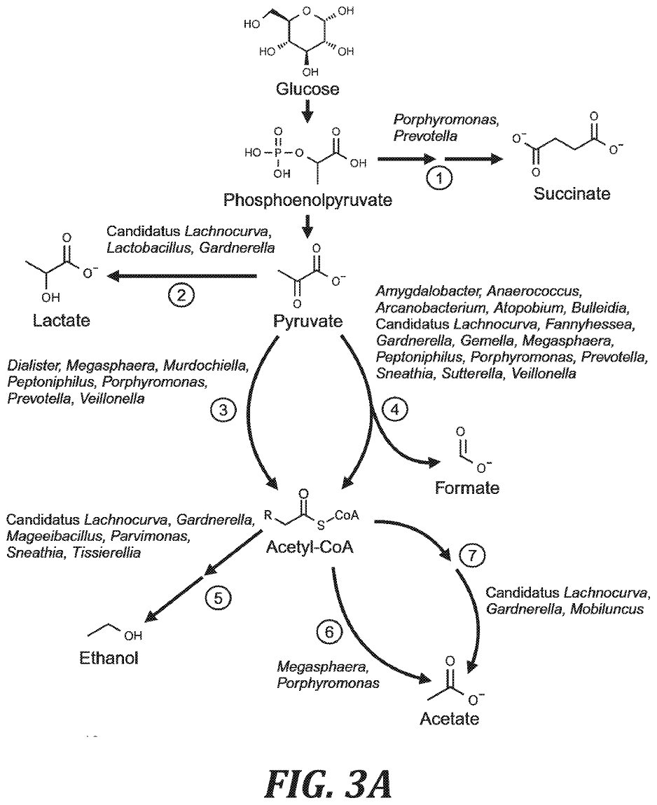

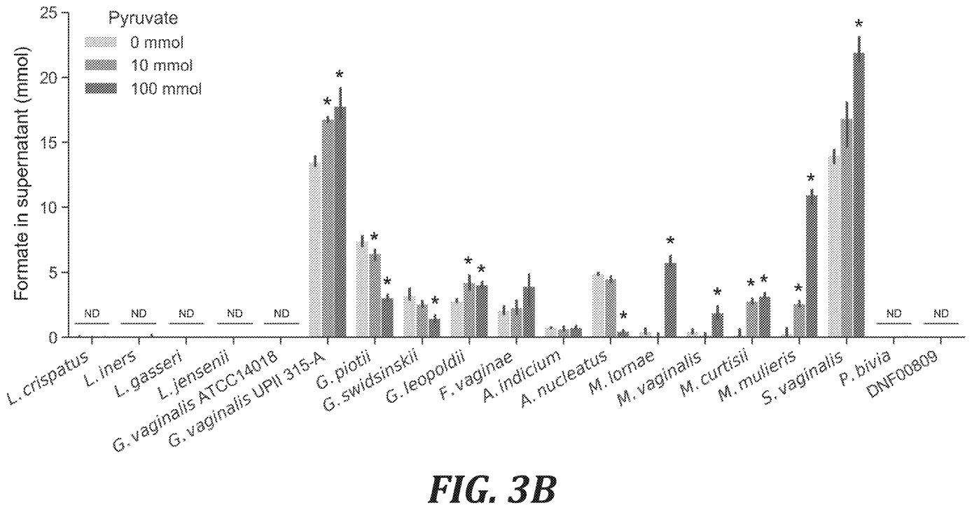

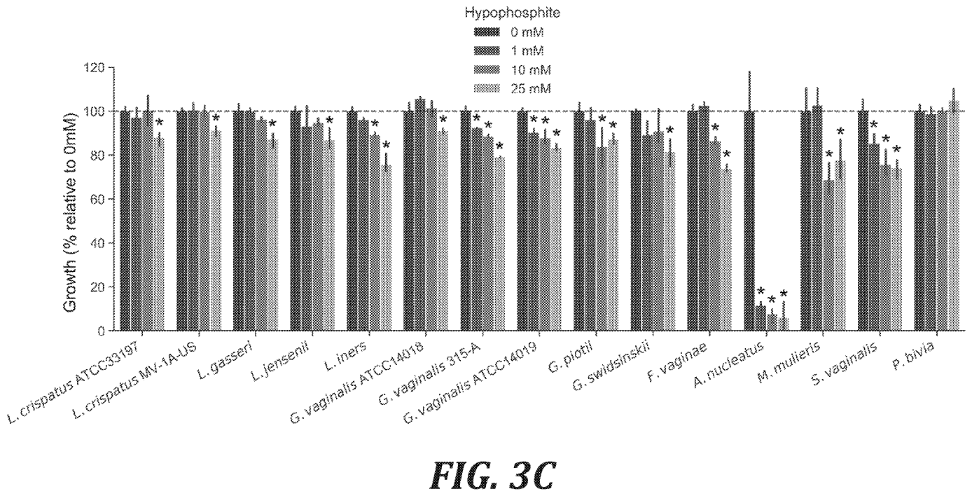

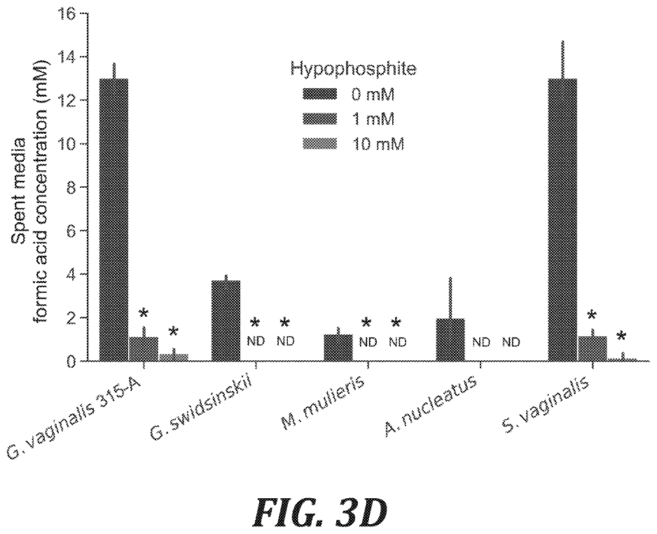

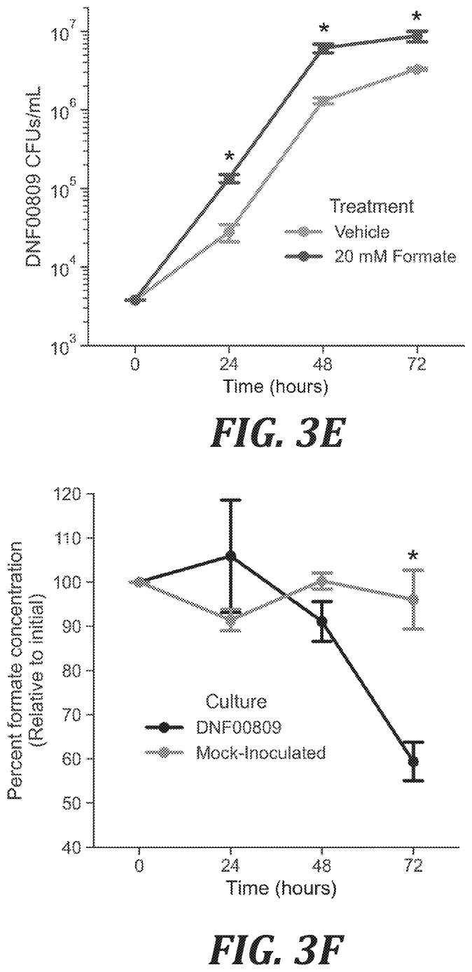

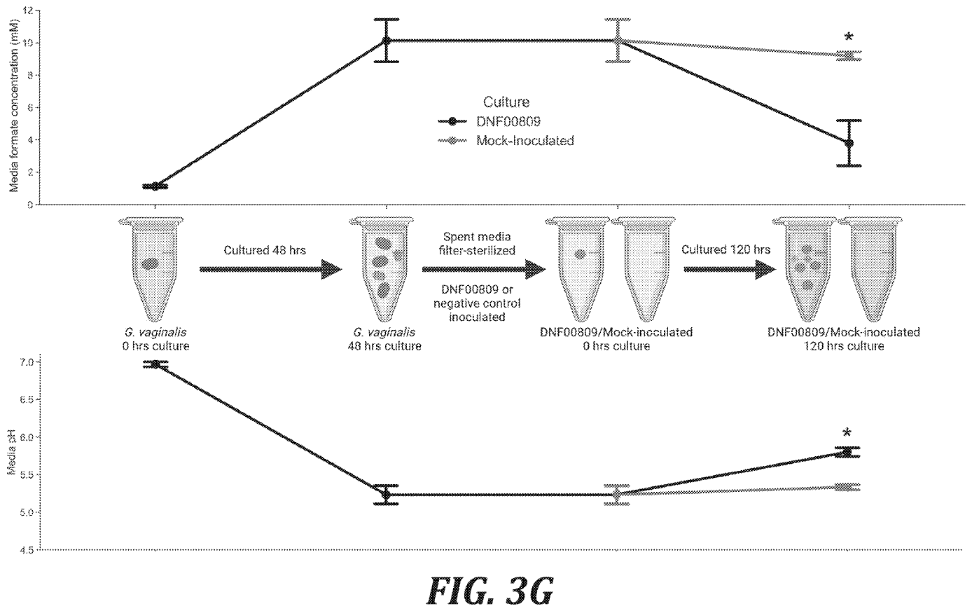

Identification of carbohydrate metabolic proteins reveals widespread formic acid fermentation among BVAB and formate utilization by Coriobacteriales bacterium DNF00809. To determine the functions performed by different organisms in vivo, the taxa associated with biologically relevant proteins identified in the present metaproteomic data were examined. Glycogen is an abundant source of carbon and energy for vaginal bacteria, but it must be broken down into smaller sugars for cells to import and metabolize. Most glycogen-degrading amylase and pullulanase enzymes exclusively matched Gardnerella proteins, but six such spectra from BV-samples matched proteins from L. crispatus (Table 2), indicating these organisms are primarily responsible for glycogen breakdown in vivo. Many sample spectra matched bacterial fermentative enzymes, revealing which taxa consumed carbohydrates released from vaginal glycogen and what metabolic end-products are likely produced (FIG. 3A, Table 2). Some spectra matched succinate dehydrogenases from Porphyromonas or Prevotella, providing evidence these organisms contribute to increased succinate concentrations associated with BV. Acetate-producing enzymes matched proteins from bacteria including Gardnerella, Megasphaera, Mobiluncus, Porphyromonas, and Candidatus Lachnocurva (alternatively known as bacterial vaginosis-associated bacterium 1 or BVAB1). Several identified proteins involved in ethanol synthesis were associated with six genera of BVAB (FIG. 3A, Table 2).

The most common bacterial fermentative enzyme in the present data was pyruvate formate-lyase (PFL) (Table 2), which converts pyruvate into formic acid and acetyl-CoA. Many of the spectra associated with this enzyme exclusively matched proteins from a single bacterial genus including 63 unique to Amygdalobacter, 52 unique to Megasphaera, and 43 unique to Gardnerella. Other PFL spectra matched proteins from many organisms, encompassing 16 different bacterial genera. Genomic analysis showed that although these enzymes are absent from commensal lactobacilli, most major BVAB encode putative PFL enzymes (Table 3). Exceptions included the Prevotella species P. amnii, P. bivia, and P. timonensis, which did not have homologs of these proteins.

To test whether or not diverse BVAB can produce formic acid, a selection of vaginal bacteria was cultured in media with varying quantities of added pyruvate, then formate present in their culture supernatants was measured. In total, twelve species of BVAB secreted detectable quantities of formic acid (FIG. 3B). Most formate-producing bacteria secreted higher concentrations when grown with supplemented pyruvate, except for Gardnerella piotii, G. swidsinskii, and Amygdalobacter nucleatus where increased pyruvate caused these isolates to grow to a lower density. Bacteria without a PFL enzyme, including four species of commensal lactobacilli, P. bivia, and Coriobacteriales bacterium DNF00809 (also named Umbribacter vaginalis) did not produce measurable quantities of formate (FIG. 3B). The type strain G. vaginalis ATCC 14018 was an outlier among this species because its PFL gene contains a premature stop codon, which was likely the reason formate was not detected in its spent culture media (FIG. 3B).

Hypophosphite is an antimicrobial and inhibitor of PFL. It was hypothesized herein that formic acid-secreting bacteria would be more sensitive to hypophosphite. In line with this hypothesis, hypophosphite concentrations as low as 1 mM significantly reduced the growth of formate-producing organisms including G. vaginalis 315-A, A. nucleatus, M. mulieris, and S. vaginalis (FIG. 3C). The effect on G. piotii, G. swidsinskii, and F. vaginae was less pronounced, but trended toward reduced growth. In contrast, hypophosphite had a much smaller effect on growth of organisms that did not secrete formic acid, including lactobacilli, P. bivia, and G. vaginalis ATCC 14018. Hypophosphite concentrations up to 10 mM did not significantly alter growth of L. crispatus, L. gasseri, or L. jensenii, but L. iners was slightly more sensitive (FIG. 3C). Formate in spent media from select organisms grown with hypophosphite was quantified and it was found that although 1 mM hypophosphite only reduced growth by 10% to 20%, this concentration nearly abolished formate secretion (FIG. 3D). G. vaginalis, G. swidsinskii, M. mulieris, A. vaginae, and S. vaginalis all produced approximately 90% lower formic acid concentrations in the presence of 1 mM hypophosphite, with stronger effects at 10 mM. These data supported hypophosphite as an efficient inhibitor of bacterial formic acid synthesis and suggested it has stronger antimicrobial properties toward organisms that rely on formate production for their growth.

Next, formate in CVL samples from a different cohort of study participants (30 BV+ and 30 BV−) was quantified to determine whether or not there was evidence of formic acid fermentation in vivo. Of these 60 samples, only 4 had formate concentrations above the assay's limit of detection, suggesting that if bacteria secrete formic acid in BV, it does not accumulate to high concentrations. Whether isolates from 7 vaginal genera could grow on formate or deplete the compound from their media was tested, but no evidence of formate consumption or increased growth on formate compared to controls was found. However, a small number of taxa including Coriobacteriales bacterium DNF00809 (Umbribacter vaginalis) encode FDH enzymes (FDHs), which may allow them to use formate as a substrate for ATP generation (Table 3). This fastidious organism is most closely related to Eggerthella and is also commonly called Eggerthella-like vaginal bacterium. Past studies have found this organism in the vaginal communities of 91% of individuals with BV, and it was present at up to 6% abundance in our samples. When DNF00809 was cultured with formate, it grew significantly more compared to control media (unpaired T-test, P<0.05) (FIG. 3E). In the same culture, this organism depleted approximately 40% of formate from its media (FIG. 3F). Coriobacteriales bacterium DNF00809 (Umbribacter vaginalis) depleted a similar percentage of formic acid from G. vaginalis spent media and significantly increased the spent media's pH (unpaired T-test, P<0.05) (FIG. 3G). Therefore, bacteria with FDHs such as Coriobacteriales bacterium DNF00809 (Umbribacter vaginalis) may serve an important role in the BV microbiota by removing formic acid secreted by other organisms.

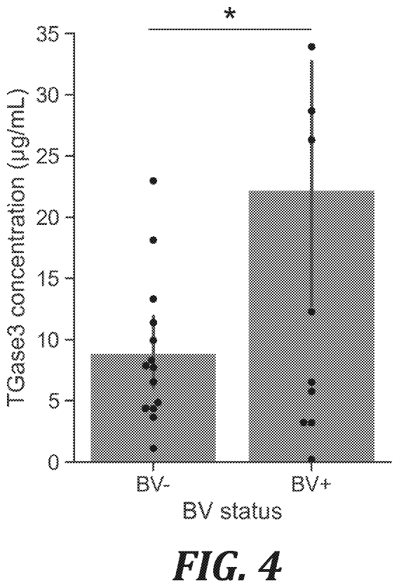

Signals of host responses to BV in metaproteomic data. Among the human proteins identified in the metaproteomic data described herein, 13 that play a role in epithelial structure had a significantly lower abundance in BV+ samples (Table 1). The human protease neutrophil elastase was observed in both BV+ and BV-samples, but its relative abundance was not different based on BV status. In contrast, the protein leukocyte elastase inhibitor had significantly lower abundance in BV+ samples, indicating host proteases may be dysregulated in BV. Although many host proteins had a lower abundance in BV+ samples, Transglutaminase 3 (TGase3) had a higher relative abundance (Table 1). Tgase3 plays a role in epithelial cornification by cross-linking proteins and can also promote wound repair by stabilizing the extracellular matrix. To knowledge of the present inventors, no previous studies have noted a role for TGase3 in the host response to BV. Thus, the concentration of TGase3 was measured in a separate set of samples to confirm or refute this metaproteomic data. Using an enzyme-linked immunosorbent assay (ELISA), the concentration of TGase3 was measured in 26 cervicovaginal fluid (CVF) samples that were not previously analyzed by metaproteomics (14 BV−, 12 BV+). In agreement with the metaproteomic data, TGase3 was detected in all 26 samples, and its concentration was found to be approximately 2× higher in CVF from BV+ participants (Welch's T-test, P=0.04) (FIG. 4).

Signs of heme and iron sequestration in BV. Iron is a vital nutrient for many pathogenic bacteria and is required for the Gardnerella to grow. In the samples used in the present example, spectra matching iron-dependent pyruvate: ferredoxin oxidoreductase were identified from a wide range of BVAB, suggesting iron is important for organisms including Prevotella, Dialister, and Megasphaera (Table 2). Also, a large number of spectra matching the human iron-binding proteins serotransferrin and lactotransferrin was identified, but the abundances of these proteins were not significantly different based on BV status. Similarly, none of the seven hemoglobin subunit proteins identified were significantly differentially abundant. However, the heme-sequestering protein hemopexin was present at a significantly higher abundance in BV+ samples, and heme-degrading heme oxygenase 1 had a significantly lower abundance (Table 1), suggesting the host may respond to BV by sequestering heme and reducing its breakdown and the concomitant release of free iron.

### Discussion

In the present example, metaproteomics was leveraged to elucidate clinically relevant bacterial processes in BV and associate them with specific taxa. D. micraerophilus was implicated in the production of the characteristic BV metabolite putrescine, and it was found that the biosynthesis of this malodorous compound can be increased by inter-species syntrophy between this organism and F. vaginae. This interaction likely relies on cross-feeding of ornithine, which is produced as a byproduct of arginine catabolismby the latter organism. Multiple malodorous amines including trimethylamine, cadaverine, and putrescine are known to be present at higher concentrations in vaginal fluid from people with BV, but it was not previously known what organisms produce these metabolites. The present results indicate that D. micraerophilus is capable of secreting relatively high concentrations of cadaverine in mono-culture. Additionally, supplementing this bacterium's culture media with ornithine or co-culturing it with F. vaginae leads to increased putrescine production. The present results also indicate that synthesis of these two polyamines may be co-regulated, because supplemental ornithine led to the total abrogation of cadaverine production. Genetic knockout experiments have not been performed to mechanistically confirm D. micraerophilus uses ODC to make putrescine, but the present findings that this organism secretes malodorous amines could be relevant to other pathologies associated with these compounds such as chronic wounds where Dialister spp. have been found. In these polymicrobial infections, related taxa may work with other anaerobic bacteria to synthesize malodorous polyamines.

It remains unclear what biological function putrescine biosynthesis serves for BVAB. Catabolizing arginine into ornithine releases ammonia and produces ATP, but converting ornithine into putrescine does not produce energy. Immune modulation may be one beneficial function for the BV microbiota because putrescine suppresses the inflammatory functions of white blood cells. Alternatively, putrescine's alkalinity may buffer the pH of vaginal fluid, making it more amenable to acid-sensitive BVAB. Putrescine is also known to inhibit the cross-linking activity of TGase3, slowing the wound-healing process. BV is strongly associated with a disrupted epithelial barrier, so putrescine may exacerbate damage done by dysregulated host and bacterial proteases. Therefore, targeting this pathway could inhibit multiple functions important for the BV microbiota and ameliorate BV-associated malodor.

Other proteins in the presently described samples that provided insights into bacterial functions were found, including extracellular glycoside hydrolases from L. crispatus in BV-samples and similar enzymes from Gardnerella in BV+ samples. Past studies suggested that human amylases were primarily responsible for releasing metabolizable sugars from vaginal glycogen, but the present data add to the growing body of evidence that vaginal bacteria are active in glycogen breakdown. The present inventors also found a higher abundance of bacterial glycosidases in BV+ samples, suggesting that competition for carbohydrates may be elevated in BV, which agrees with past reports of lowered carbohydrate concentrations associated with the condition. The present results also suggest the microbiota is more metabolically active in BV, which is concordant with the present qPCR data and other studies that have found higher concentrations of bacteria in individuals with BV.

The metaproteomic data provided insights into bacterial fermentation, showing PFLs are expressed in the vagina by up to 16 genera of BVAB. This observation was unexpected because formic acid and lactic acid have similar pKa values and BVAB are generally more pH-sensitive than commensal lactobacilli. However, the present inventors subsequently confirmed that 12 species of BVAB can produce high concentrations of formic acid in vitro. The present inventors also confirmed that hypophosphite, a PFL inhibitor and food additive, which is generally regarded as safe by the U.S. Food and Drug Administration, severely inhibits formic acid secretion by diverse BVAB and has a stronger antimicrobial effect on formate-producing BVAB between 1 mM to 10 mM than L. crispatus, L. gasseri, and L. jensenii. Although these results suggest that formic acid fermentation could be widespread in the BV microbiota, only 4 of 60 CVF samples tested as described herein had detectable concentrations of formate (>130 nM). These measurements were in-line with a previous proton nuclear magnetic resonance (1H-NMR) study that confirmed formic acid is elevated in CVL from individuals with BV, but present at low concentrations (34 nM in BV−, 450 nM in BV+). These in vivo results imply that if bacteria in the BV microbiota secrete formic acid, most of it is quickly consumed. The present results implicate Coriobacteriales bacterium DNF00809, recently named Umbribacter vaginalis, as one of the organisms which could perform this function. This fastidious, slow-growing bacterium is highly prevalent in BV and its genome contains homologs of FDH enzymes that may allow it to metabolize formate for ATP generation. Indeed, DNF00809 (Umbribacter vaginalis) grew more efficiently in the presence of formate and depleted both exogenously added and BVAB-secreted formate from its culture media. DNF00809 (Umbribacter vaginalis) also significantly increased the pH of spent G. vaginalis media. Although it is unclear whether DNF00809 (Umbribacter vaginalis) reduced the acidity of spent media primarily by depleting formic acid or through other behaviors such as NH₄⁺ secretion, these results raise the possibility that minority taxa could play an important role in BV by consuming organic acids secreted by higher-abundance taxa such as Gardnerella or Sneathia, thereby buffering the environment and allowing other bacteria to metabolize more host-derived nutrients. Additional research will be required to determine how prevalent formic acid fermentation is among BVAB in vivo and whether other FDH-encoding organisms such as Finegoldia magna can fulfill a similar function.

The human proteins in the presently described dataset provide insights into potential host responses to BV. Although the iron-binding proteins serotransferrin and lactotransferrin are known to play a role in innate immune defense by sequestering iron, no significant differences were observed in either of these proteins. Two heme-associated proteins were differentially abundant, however. The heme-sequestering protein hemopexin was present at a higher relative abundance in BV+ samples. In contrast, heme oxygenase 1 was present at a lower relative abundance in BV+ samples. This enzyme degrades heme, releasing iron. Together, these data imply the host may not respond to BV by significantly changing expression of iron-binding transferrins, but may instead respond by sequestering heme away from bacteria and reducing the breakdown of this molecule along with the release of free iron.

A strength of metaproteomic analysis is that proteins are sampled and identified from a community in its natural state, therefore inferences can be drawn about microbial functions in their normal biological context. This is especially useful in the context of the vaginal microbiota where the dearth of representative model systems makes it difficult to experimentally manipulate human-associated microorganisms. These insights are only inferential, however, and must be complemented with rigorous experimentation to draw conclusions. The in vitro culture experiments described herein supported the present inventors' hypotheses, based on metaproteomic data, that diverse BVAB secrete formate and a syntrophic relationship could exist between D. micraerophilus and F. vaginae to produce putrescine. However, these data provide evidence that bacteria are capable of these functions, not that they necessarily perform them in vivo. Further studies that leverage systems that better mimic the vaginal microenvironment, such as biofilm flow systems or ex vivo organotypic models, are required to better elucidate how formate metabolism and putrescine biosynthesis contribute to bacterial colonization of the vagina.

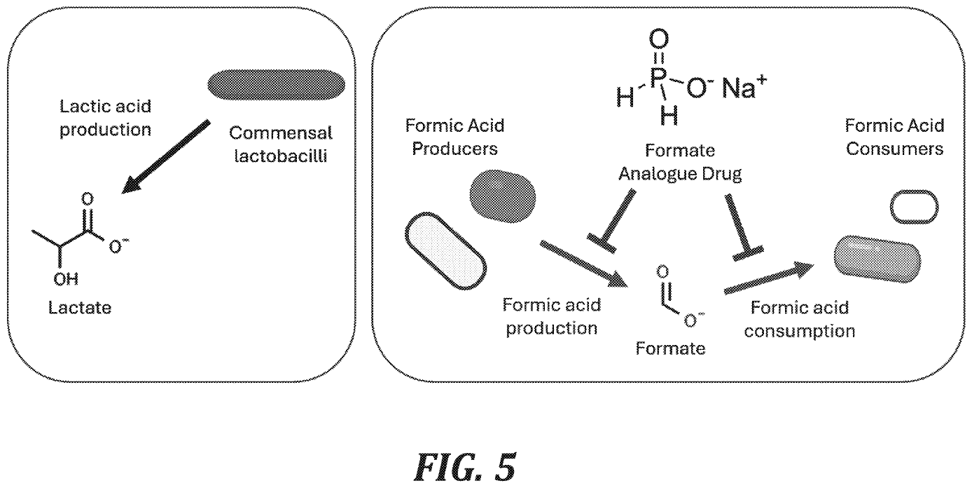

The present example demonstrates the value of analyzing metaproteomic data at different scales. Broad, high-level analysis enables the identification of general functional differences between microbial communities, whereas parsing individual protein identifications can uncover biologically relevant functions performed by different organisms in deeper analyses. The present inventors identified signs of increased, diversified bacterial metabolism in BV, along with potential host responses to epithelial barrier damage. The bacterial proteins identified herein provide evidence for which taxa are responsible for characteristic BV metabolites such as acetate, succinate, and trimethylamine, and suggest cross-feeding interactions based on glutamate. Besides glycolytic enzymes, the most abundant bacterial peptide identified in BV samples was linked to formate metabolism, revealing an unexpected and wide-spread metabolic pathway in BVAB. Further experiments revealed Coriobacteriales bacterium DNF00809 (Umbribacter vaginalis) is capable of metabolizing formic acid produced by other BVAB. A new, syntrophic interaction between D. micraerophilus and F. vaginae that can increase putrescine biosynthesis was also discovered. These proteins represent targets for novel treatments that could address BV symptoms and disrupt metabolic pathways that are critical to the BV microbiota. For example, broad-spectrum protease inhibitors may reduce the activity of both host and bacterial proteases, starving BVAB of amino acids. More targeted interventions, such as small molecule inhibitors of GrdH and ODC could reduce synthesis of TMA and putrescine and alleviate malodor. PFLs are an especially promising target because these enzymes are encoded by a wide range of BVAB but are completely absent from commensal lactobacilli. 10 mM of the PFL inhibitor sodium hypophosphite was shown to severely reduce bacterial formic acid secretion and exert a stronger antimicrobial effect on formate-producing BVAB such as Gardnerella, Sneathia, and Amygdalobacter than most commensal lactobacilli. Specifically designed small molecule inhibitors of these enzymes could more efficiently disrupt carbohydrate metabolism in BVAB without harming commensal lactobacilli (FIG. 5). Such interventions could supplement or replace existing BV treatments, reduce recurrences, and prevent BV in those at highest risk.

### Tables

TABLE 1
Significantly differentially abundant proteins by BV status

| | Host or | Higher/Lower | |
|---|---|---|---|
| Protein | Bacterial | Abundance in BV | Function Category |
| Fructose-bisphosphate aldolase | Bacterial | Lower | Glycolysis |
| Pyruvate kinase | Bacterial | Lower | Glycolysis |
| Enolase | Bacterial | Lower | Glycolysis |
| Phosphoglycerate kinase | Bacterial | Lower | Glycolysis |
| 6-phosphofructokinase | Bacterial | Lower | Glycolysis |
| Glyceraldehyde-3-phosphate | Bacterial | Lower | Glycolysis |
| dehydrogenase | | | |
| Phosphoglycerate mutase | Bacterial | Lower | Glycolysis |
| Phosphoenolpyruvate carboxykinase | Bacterial | Higher | Gluconeogenesis |
| Pyruvate, phosphate dikinase | Bacterial | Higher | Gluconeogenesis |
| Lactic acid dehydrogenase | Bacterial | Lower | Lactate Fermentation |
| Pyruvate formate lyase | Bacterial | Higher | Formate Fermentation |
| Alcohol dehydrogenase | Bacterial | Higher | Alcohol Fermentation |
| Acetate kinase | Bacterial | Higher | Acetate Fermentation |
| Extracellular glycosidase | Bacterial | Higher | Polysaccharide Breakdown |
| Glutamate dehydrogenase | Bacterial | Higher | Nitrogen Metabolism |
| Glutamate transporter | Bacterial | Higher | Nitrogen Metabolism |
| Glycine reductase | Bacterial | Higher | Amino Acid Metabolism |
| Glutamine synthase | Bacterial | Higher | Amino Acid Metabolism |
| Ornithine transcarbamoylase | Bacterial | Higher | Amino Acid Metabolism |
| Extracellular Peptidase | Bacterial | Higher | Protein Breakdown |
| RNA Polymerase | Bacterial | Higher | General Metabolism |
| ATP synthase | Bacterial | Higher | General Metabolism |
| ABC transporter | Bacterial | Higher | General Metabolism |
| Ribosome | Bacterial | Higher | General Metabolism |
| Peroxiredoxin | Bacterial | Higher | Antioxidant |
| Ladinin-1 | Host | Lower | Epithelial Structure |
| Catenin alpha-2 | Host | Lower | Epithelial Structure |
| Involucrin | Host | Lower | Epithelial Structure |
| SPR3 | Host | Lower | Epithelial Structure |
| Cornifelin | Host | Lower | Epithelial Structure |
| Protein S100-A10 | Host | Lower | Epithelial Structure |
| Tight junction protein ZO-1 | Host | Lower | Epithelial Structure |
| Cornulin | Host | Lower | Epithelial Structure |
| Periplakin | Host | Lower | Epithelial Structure |
| Zyxin | Host | Lower | Epithelial Structure |
| Filaggrin | Host | Lower | Epithelial Structure |
| Cadherin-1 | Host | Higher | Epithelial Structure |
| Cornifin-A | Host | Lower | Epithelial Structure |
| S100-A11 | Host | Lower | Epithelial Structure |
| Transglutaminase 3 | Host | Higher | Epithelial Repair |
| Pyruvate kinase PKLR | Host | Lower | Glycolysis |
| Glyceraldehyde-3-phosphate | Host | Lower | Glycolysis |
| dehydrogenase | | | |
| Phosphoglycerate kinase 2 | Host | Lower | Glycolysis |
| Alpha-enolase | Host | Lower | Glycolysis |
| ATP-dependent 6- | Host | Lower | Glycolysis |
| phosphofructokinase, platelet type | | | |
| Glucose-6-phosphate isomerase | Host | Lower | Glycolysis |
| ATP-dependent 6- | Host | Lower | Glycolysis |
| phosphofructokinase, liver type | | | |
| L-lactate dehydrogenase A chain | Host | Lower | Acidification |
| Glycogen phosphorylase, liver form | Host | Lower | Glycogen Metabolism |
| Leukocyte elastase inhibitor | Host | Lower | Protease Inhibitor |
| Calpastatin | Host | Lower | Protease Inhibitor |
| Cystatin-A | Host | Lower | Protease Inhibitor |
| Serpin B4 | Host | Lower | Protease Inhibitor |
| Serpin B5 | Host | Lower | Protease Inhibitor |
| Cystatin-B | Host | Lower | Protease Inhibitor |
| Serpin B3 | Host | Lower | Protease Inhibitor |
| Serpin I2 | Host | Lower | Protease inhibitor |
| Hemopexin | Host | Higher | Heme Sequestration |
| Heme oxygenase 1 | Host | Lower | Heme Breakdown |

Significantly differentially abundant proteins as determined by Mann-Whitney U Test (P<0.05) from the host or bacteria. Homologous proteins from different bacterial species were identified and grouped using eggnog-mapper.

TABLE 2
Biologically relevant bacterial proteins identified in CVL samples

| | | BV− Total | BV+ Total |
|---|---|---|---|
| | | Spectral | Spectral |
| Protein | Associated Taxa | Count | Count |
| Type I Pullulanase | Gardnerella | 0 | 32 |
| Secreted Alpha-Amylase | Gardnerella | 0 | 40 |
| Type I Pullulanase | Lactobacillus crispatus | 6 | 0 |
| Pyruvate Formate-Lyase | 16 BVAB Genera^{a} | 0 | 180 |
| Pyruvate Formate-Lyase | Amygdalobacter (BVAB2) | 0 | 63 |
| Pyruvate Formate-Lyase | Megasphaera | 0 | 52 |
| Pyruvate Formate-Lyase | Gardnerella | 0 | 43 |
| Anaerobic Ribonucleoside- | Gardnerella | 31 | 119 |
| Triphosphate Reductase | | | |
| Lactate Dehydrogenase | Lactobacillus | 168 | 37 |
| Lactate Dehydrogenase | Gardnerella | 0 | 49 |
| Lactate Dehydrogenase | Candidatus Lachnocurva | 0 | 8 |
| | (BVAB1) | | |
| Phosphate Acetyltransferase | Candidatus Lachnocurva | 0 | 5 |
| | (BVAB1) | | |
| Phosphate Acetyltransferase | Gardnerella | 0 | 52 |
| Acetate Kinase | Gardnerella | 0 | 32 |
| Acetate Kinase | Gardnerella, Mobiluncus | 1 | 41 |
| Acetyl-CoA Hydrolase | Megasphaera, | 0 | 14 |
| | Porphyromonas | | |
| Phosphoenolpyruvate | Porphyromonas, | 0 | 190 |
| Carboxykinase | Prevotella | | |
| Malate Dehydrogenase | Prevotella | 0 | 41 |
| Fumarate Hydratase | Prevotella | 0 | 4 |
| Succinate Dehydrogenase | Porphyromonas, | 0 | 14 |
| | Prevotella | | |
| Alcohol Dehydrogenase | Gardnerella | 0 | 72 |
| Alcohol Dehydrogenase | Candidatus Lachnocurva | 3 | 68 |
| | (BVAB1), Gardnerella, | | |
| | Mageeibacillus, | | |
| | Parvimonas, Sneathia, | | |
| | Tissierellia | | |
| Pyruvate:Ferredoxin | Dialister, Megasphaera, | 0 | 80 |
| Oxidoreductase | Murdochiella, | | |
| | Peptoniphilus, | | |
| | Porphyromonas, | | |
| | Prevotella, Veillonella | | |
| Glutamate Dehydrogenase | Aerococcus, | 0 | 167 |
| | Arcanobacterium, | | |
| | Fannyhessea, Gemella, | | |
| | Megasphaera, | | |
| | Mobiluncus, | | |
| | Porphyromonas, | | |
| | Prevotella, Streptococcus | | |
| Glutamate Transporter | Gardnerella | 0 | 32 |
| Glutamine Synthetase | Gardnerella | 0 | 33 |
| Arginine Deiminase | Fannyhessea vaginae | 0 | 6 |
| Arginine Deiminase | Sneathia vaginalis | 0 | 8 |
| Ornithine Carbamoyltransferase | Fannyhessea, Sneathia, | 0 | 11 |
| | Mageeibacillus, | | |
| | Parvimonas | | |
| Ornithine Decarboxylase | Dialister micraerophilus | 0 | 9 |

Bacterial proteins with potential biological significance identified in CVL samples. All taxa associated with proteins matching the sample spectra are listed, along with total spectral count across all BV− or BV+ samples. ^{a}The 16 genera of BVAB associated with pyruvate formate-lyase were Amygdalobacter (BVAB2), Anaerococcus, Arcanobacterium, Atopobium, Bulleidia, Candidatus Lachnocurva (BVAB1), Fannyhessea, Gardnerella, Gemella, Megasphaera, Peptoniphilus, Porphyromonas, Prevotella, Sneathia, Sutterella, and Veillonella. Spectra that exclusively matched proteins from a single genus are listed on separate rows.

TABLE 3
Distribution of key metabolic enzymes in vaginal bacteria

| | Formate | | | Glutamate | | | TMA | | Putrescine | | |
|---|---|---|---|---|---|---|---|---|---|---|---|
| | PFL | FDH | NrdD | GlnA | GdhA | GluA | TorA | GrdA | ADI | OCT | ODC |
| Lactobacillus | | | ✓ | ✓ | | ✓ | | | ✓ | | ✓ |
| crispatus | | | | | | | | | | | |
| Lactobacillus | | | ✓ | ✓ | | ✓ | | | | | |
| iners | | | | | | | | | | | |
| Lactobacillus | | | ✓ | ✓ | | ✓ | | | | | ✓ |
| gasseri | | | | | | | | | | | |
| Lactobacillus | | | ✓ | ✓ | | ✓ | | | ✓ | ✓ | ✓ |
| jensenii | | | | | | | | | | | |
| Amygdalobacter | ✓ | | ✓ | ✓ | | | | ✓ | ✓ | ✓ | |
| indicium | | | | | | | | | | | |
| (BVAB2) | | | | | | | | | | | |
| Amygdalobacter | ✓ | | ✓ | ✓ | | | | ✓ | ✓ | ✓ | |
| nucleatus | | | | | | | | | | | |
| Candidatus | ✓ | | ✓ | | ✓ | ✓ | | | ✓ | ✓ | |
| Lachnocurva | | | | | | | | | | | |
| (BVAB1) | | | | | | | | | | | |
| Coriobacteriales | | ✓ | ✓ | ✓ | ✓ | | ✓ | | | ✓ | |
| bacterium | | | | | | | | | | | |
| DNF00809 | | | | | | | | | | | |
| (Umbribacter | | | | | | | | | | | |
| vaginalis) | | | | | | | | | | | |
| Dialister | | | ✓ | ✓ | | ✓ | | | | | ✓ |
| micraerophilus | | | | | | | | | | | |
| Fannyhessea | ✓ | | ✓ | ✓ | ✓ | ✓ | | | ✓ | ✓ | |
| vaginae | | | | | | | | | | | |
| Finegoldia magna | ✓ | ✓ | ✓ | | ✓ | | ✓ | ✓ | ✓ | ✓ | |
| Gardnerella | ✓ | | ✓ | ✓ | | ✓ | | | | | |
| Mageeibacillus | ✓ | | ✓ | | | ✓ | | | ✓ | ✓ | |
| indolicus | | | | | | | | | | | |
| Megasphaera | ✓ | | ✓ | ✓ | ✓ | ✓ | | | | | |
| hutchinsoni | | | | | | | | | | | |
| Megasphaera | ✓ | ✓ | ✓ | ✓ | ✓ | ✓ | ✓ | ✓ | | ✓ | ✓ |
| lornae | | | | | | | | | | | |
| Mobiluncus | ✓ | ✓ | | ✓ | ✓ | | ✓ | | ✓ | | |
| curtisii | | | | | | | | | | | |
| Mobiluncus | ✓ | ✓ | | ✓ | ✓ | | | | | ✓ | |
| mulieris | | | | | | | | | | | |
| Parvimonas micra | ✓ | ✓ | | | ✓ | | | ✓ | ✓ | ✓ | |
| Peptoniphilus | ✓ | | ✓ | ✓ | ✓ | ✓ | | | ✓ | | |
| Porphyromonas | ✓ | | ✓ | ✓ | ✓ | | | | | | |
| Prevotella amnii | | | | ✓ | ✓ | | | | | | |
| Prevotella bivia | | | | ✓ | ✓ | | | | | | |
| Prevotella | | | ✓ | ✓ | ✓ | | | | | | |
| timonensis | | | | | | | | | | | |
| Sneathia vaginalis | ✓ | | ✓ | | | | | | ✓ | ✓ | |
| TM7 | | ✓ | ✓ | ✓ | ✓ | | | | | ✓ | |

Presence of a homologous protein for a given bacterial species is represented by a check mark. The cell is empty for species with no significant matches to the query protein. BLASTp searches for reference proteins were performed against all bacterial proteins used to build the sequence databases described herein. A protein was determined to be present in a bacterial species if at least one isolate encoded a homolog for the query sequence with an E-value <0.0001. Proteins are grouped by the metabolite associated with their enzymatic activity. Proteins are PFL: pyruvate formate-lyase, FDH: formate dehydrogenase, NrdD: anaerobic ribonucleoside-triphosphate reductase, GlnA: glutamine synthetase, GdhA: glutamate dehydrogenase, GluA: glutamate transporter, TorA: trimethylamine-N-oxide reductase, GrdA: betaine reductase, ADI: arginine deiminase, OCT: ornithine carbamoyltransferase, ODC: ornithine decarboxylase.

### Data Availability

Protein sequence database files, metaproteomic search result files, and genes identified by shotgun metagenomic sequencing used and described in the present example are available at the figshare.com website under “Project Database and Result Files” and identified by DOI: 10.6084/m9.figshare.25705839.v1. Code files used for data analysis and figure generation are available at the zenodo.org website under reference number 11075582. Raw mass spectrometry data generated in the present example have been deposited to the ProteomeXchange Consortium via the PRIDE partner repository with the database identifier PXD051980.

While illustrative embodiments have been described, it will be appreciated that various changes can be made therein without departing from the spirit and scope of the disclosure. The present disclosure is not to be limited in scope by the specific embodiments described herein. Indeed, various modifications of the invention, in addition to those described herein, will become apparent to those skilled in the art from the foregoing description and the accompanying figures. Such modifications are intended to fall within the scope of the appended claims. The disclosure is, therefore, to be limited only by the terms of the appended claims along with the full scope of equivalents to which the claims are entitled.

The embodiments of the invention in which an exclusive property or privilege is claimed are defined as follows:

1. A method of treating bacterial vaginosis (BV) or reducing a recurrence of BV in a subject, the method comprising administering to the subject a therapeutically effective amount of an inhibitor of formic acid metabolism in one or more BV-associated bacteria, wherein the inhibitor prevents or reduces growth, metabolic activity, or a combination thereof, of the one or more BV-associated bacteria.

2. The method of claim 1, wherein the therapeutically effective amount is an amount effective to inhibit formic acid production, formic acid consumption, or a combination thereof, by the one or more BV-associated bacteria.

3. The method of claim 1, wherein the inhibitor is an inhibitor of pyruvate formate lyase (PFL).

4. The method of claim 1, wherein the inhibitor is an inhibitor of formate dehydrogenase (FDH).

5. The method of claim 1, wherein the inhibitor comprises a formate analog.

6. The method of claim 1, wherein the inhibitor comprises hypophosphite or a pharmaceutically acceptable salt thereof.

7. The method of claim 1, wherein the one or more BV-associated bacteria comprise Gardnerella vaginalis, Gardnerella piotii, Gardnerella leopoldii, Gardnerella swidsinskii, Fannyhessea vaginae, Amygdalobacter indicium, Amygdalobacter nucleatus, Megasphaera lornae, Megasphaera vaginalis, Megasphaera curtisii, Megasphaera mulieris, Prevotella bivia, Sneathia vaginalis, Umbribacter vaginalis, or a combination thereof.

8. The method of claim 1, wherein the inhibitor selectively prevents or reduces growth, metabolic activity, or a combination thereof, of the one or more BV-associated bacteria relative to one or more Lactobacillus species.

9. The method of claim 8, wherein the inhibitor does not impact a growth of the one or more Lactobacillus species.

10. The method of claim 8, wherein the one or more Lactobacillus species comprise Lactobacillus crispatus, Lactobacillus jensenii, Lactobacillus gasseri, Lactobacillus iners, or a combination thereof.

11. The method of claim 1, wherein the inhibitor is formulated as a topical composition for intravaginal application.

12. The method of claim 1, wherein the inhibitor is administered intravaginally.

13. The method of claim 1, wherein the inhibitor is administered in combination with an antibiotic for treating BV.

14. The method of claim 1, wherein the inhibitor is administered after completion of an antibiotic treatment for BV.

15. The method of claim 1, wherein the inhibitor is administered prophylactically to a subject at risk of BV recurrence.

16. The method of claim 1, wherein administration of the inhibitor promotes restoration of a vaginal microbiota dominated by one or more Lactobacillus species.

17. The method of claim 1, wherein administration of the inhibitor reduces vaginal malodor associated with BV.
